(19) United States
(12) Patent Application Publication
(10) Pub. No.: US 2010/0144872 A1
(43) Pub. Date: Jun. 10, 2010

(54) New Methylenebisphenyl Compounds Useful in the Treatment of Inflammation

(75) Inventors: Benjamin Pelcman, Solna (SE); Peter Nilsson, Solna (SE)

Correspondence Address: K&L Gates LLP, 1900 MAIN STREET, SUITE 600, IRVINE, CA, 92614-7319, US

(73) Assignee: Biolipox AB

(21) Appl. No.: 12/529,913

(22) PCT Filed: Mar. 4, 2008
(86) PCT No.: PCT/GB2008/000724

§ 371 Date: Feb. 26, 2010

Related U.S. Application Data
(60) Provisional application No. 60/904,784, filed on Mar. 5, 2007.
(60) Provisional application No. 60/904,783, filed on Mar. 5, 2007.

(51) Int. Cl. A61K 31/235; C07C 69/76; C07C 63/331; C07C 229/00; A61K 31/192; A61K 31/195; A61P 11/00; A61P 29/00

(52) U.S. Cl. 514/533; 560/76; 562/488; 562/441; 514/568

(57) ABSTRACT

There is provided compounds of formula (I), wherein R^{x}, R^{y}, X¹, X², L¹, L², Y¹ and Y² have meanings given in the description, and pharmaceutically-acceptable salts thereof, which compounds are useful in the treatment of diseases in which inhibition of the activity of leukotriene C₄ synthase is desired and/or required, and particularly in the treatment of a respiratory disorder and/or inflammation.

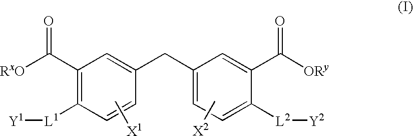

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. It may be converted, e.g. in the event of inflammation, into mediators, some of which are known to have beneficial properties and others that are harmful. Such mediators include leukotrienes (formed by the action of 5-lipoxygenase (5-LO), which acts by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRAs) have been developed for the treatment of asthma, but they are often highly selective for CysLT₁. It may be hypothesised that better control of asthma, and possibly also COPD, may be attained if the activity of both of the CysLT receptors could be reduced. This may be achieved by developing unselective LTRAs, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén J. Internal Med. 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialized world. In children, the incidence is even higher, being close to 10% in most countries. Asthma is the most common cause of hospitalization for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRAs have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:
(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

International patent application WO 2005/092836 discloses various biaryl compounds. However, such compounds are only described as being useful opioid receptor antagonists, and therefore useful in the treatment of obesity and related diseases such as diabetes.

International patent application WO 2007/113337 discloses various biaryl sulfonamides. However, such compounds are only described as being useful fluorescent markers of proteins, for use in high throughput screening tests.

International patent application WO 2005/083081 discloses a biaryl sulphonamide that is useful as a nuclease inhibitor.

International patent application WO 2004/076640 discloses a biaryl sulfonamide compound that is useful as an inhibitor of angiogenin and RNases.

Kao, R. Y. T., et al., P. Natl. Acad. Sci. USA, 2002, 99(15), 10066-10071 and Jenkins, J. L., Protein, 2003, 50, 81-93 both disclose various biaryl sulfonamides as angiogenin inhibitors.

U.S. Pat. No. 2,438,782 and U.S. Pat. No. 2,435,629 and UK patent No. GB 577,387 all disclose biaryl sulfonamides useful as, or in the synthesis of, photographic dyes.

Finally, Li, J. et al., Bioorg. Med. Chem., 2006, 14, 2209-2224 discloses various biaryl compounds that are purportedly useful as inhibitors of human cyclophilin A.

There is no disclosure in any of the prior art of derivatives of 5,5′-methylenebis(2-aminobenzoic acid) for use as LTC₄ synthase inhibitors, and therefore for use in the treatment of inflammation or respiratory disorders.

### DISCLOSURE OF THE INVENTION

According to the invention, there is provided a compound of formula I,

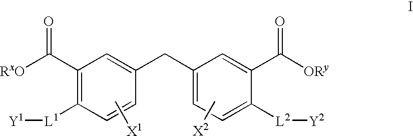

wherein
Y¹ represents H or —Ar¹;
Y² represents H or —Ar²;
provided that at least one of Y¹ and Y² is other than H;
X¹ and X² independently represent one or more optional substituents selected from halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4d}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} and —OS(O)₂N(R⁴ⁱ)R⁵ⁱ;
m represents 0, 1 or 2;
R^{3b} to R^{3h}, R^{3j}, R^{3k}, R³ⁿ, R^{4a} to R⁴ⁱ, R^{5a}, R^{5b}, R^{5d} and R^{5f} to R⁵ⁱ independently represent H or R^{3a}; or
any of the pairs R^{4a} and R^{5a}, R^{4b} and R^{5b}, R^{4d} and R^{5d}, R^{4f} and R^{5f}, R^{4g} and R^{5g}, R^{4h} and R^{5h} or R⁴ⁱ and R⁵ⁱ may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or R^{3a};
R³ⁱ, R^{3m} and R^{3p} independently represent R^{3a};
R^{3a} represents, on each occasion when mentioned above, C₁₋₆ alkyl optionally substituted by one or more substituents selected from F, Cl, —CN, —N₃, ═O, —OR^{6a}, —N(R^{6b})R^{7b}, —S(O)ₙR^{6c}, —S(O)₂N(R^{6d})R^{7d} or —OS(O)₂N(R^{6e})R^{7e};
n represents 0, 1 or 2;
R^{6a}, R^{6b}, R^{6c}; R^{6d} and R^{6e} independently represent H or C₁₋₆ alkyl optionally substituted by one or more substituents selected from F, Cl, ═O, —OR^{8a}, —N(R^{9a})R^{10a} or —S(O)₂-M¹;
R^{7b}, R^{7d} and R^{7e} independently represent H, —S(O)₂CH₃, —S(O)₂CF₃ or C₁₋₆ alkyl optionally substituted by one or more substituents selected from F, Cl, ═O, —OR^{11a}, —N(R^{12a})R^{13a} or —S(O)₂-M²; or
R^{6b} and R^{7b}, R^{6d} and R^{7d} or R^{6e} and R^{7e} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or C₁₋₃ alkyl optionally substituted by one or more substituents selected from ═O and fluoro;
M¹ and M² independently represent —CH₃, —CH₂CH₃, —CF₃ or —N(R^{14a})R^{15a};
R^{8a} and R^{11a} independently represent H, —CH₃, —CH₂CH₃, —CF₃ or —CHF₂;
R^{9a}, R^{10a}, R^{12a}, R^{13a}, R^{14a} and R^{15a} independently represent H, —CH₃ or —CH₂CH₃,
Ar¹ and Ar² independently represent an aryl group or a heteroaryl group, both of which groups are optionally substituted by one or more substituents selected from A;

A represents, on each occasion when mentioned above:

I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;

G¹ represents, on each occasion when mentioned above, halo, cyano, —N₃, —NO₂, —ONO₂ or -A¹-R^{16a};

wherein A¹ represents a single bond or a spacer group selected from —C(O)A²-, —S—, —S(O)₂A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ independently represent a single bond, —C(O)—, —C(O)N(R^{17d})—, —C(O)O—, —S(O)₂— or —S(O)₂N(R^{17e})—;

Z¹ represents, on each occasion when mentioned above, ═O, ═S, ═NOR^{16b}, ═NS(O)₂N(R^{17f})R^{16c}, ═NCN or ═C(H)NO₂;

B represents, on each occasion when mentioned above:

I) an aryl group or a heteroaryl group, both of which are optionally substituted by one or more substituents selected from G²;
II) C₁₋₈ alkyl or a heterocycloalkyl group, both of which are optionally substituted by one or more substituents selected from G² and/or Z²; or
III) a G² group;

G² represents, on each occasion when mentioned above, halo, cyano, —N₃, —NO₂, —ONO₂ or -A⁶-R^{18a};

wherein A⁶ represents a single bond or a spacer group selected from —C(O)A⁷-, —S—, —S(O)₂A⁸-, —N(R^{19a})A⁹- or —OA¹⁰-, in which:

A⁷ represents a single bond, —O—, —N(R^{19b})— or —C(O)—;
A⁸ represents a single bond, —O— or —N(R^{19c})—;
A⁹ and A¹⁰ independently represent a single bond, —C(O)—, —C(O)N(R^{19d})—, —C(O)O—, —S(O)₂— or —S(O)₂N(R^{19e})—;
Z² represents, on each occasion when mentioned above, ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, for example when present on the same or on adjacent atoms, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;

G³ represents, on each occasion when mentioned above, halo, cyano, —N₃, —NO₂, —ONO₂ or -A¹¹-R^{20a};

wherein A¹¹ represents a single bond or a spacer group selected from —C(O)A¹²-, —S—, —S(O)₂A¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ independently represent a single bond, —C(O)—, —C(O)N(R^{21d})—, —C(O)O—, —S(O)₂— or —S(O)₂N(R^{21e})—;

Z³ represents, on each occasion when mentioned above, ═O, ═S, ═NOR^{20b}, ═NS(O)₂N(R^{21f})R^{20c}, ═NCN or ═C(H)NO₂;

R^{20a}, R^{20b}, R^{20c}, R^{21a}, R^{21b}, R^{21c}, R^{21d}, R^{21e} and R^{21f} are independently selected from:

i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may, for example when present on the same or on adjacent atoms, be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;

L¹ represents —N(R^{w})A¹⁹-;

L² represents —N(R^{z})A²⁰-;

A¹⁹ represents a single bond, —C(O)N(R^{w})—, —S(O)₂— or —CH₂—;

A²⁰ represents a single bond, —C(O)N(R^{z})—, —S(O)₂— or —CH₂—;

provided that when A¹⁹ represents —S(O)₂—, Y¹ represents Ar¹, and when A²⁰ represents —S(O)₂—, then Y² represents Ar²;

R^{x}, R^{y}, R^{w} and R^{z} independently represent, on each occasion when used herein, H, C₁₋₁₄ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d}));

R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} are independently selected from hydrogen and C₁₋₄ alkyl, which latter group is optionally substituted by one or more substituents selected from fluoro, chloro or ═O,

or a pharmaceutically-acceptable salt thereof,

and further provided that, when X¹ and X² are not present, and:
(a) R^{x} and R^{y} independently represent H or methyl and L¹ and L² both represent —N(H)—CH₂—, then Ar¹ and Ar² do not both represent unsubstituted phenyl;
(b) R^{x} and R^{y} independently represent H or methyl optionally substituted by unsubstituted phenyl, or one of R^{x} and R^{y} represents H and the other represents methyl, and L¹ and L² both represent —N(H)—S(O)₂—, then Ar¹ and Ar² do not both represent 4-methylphenyl; and
(c) when R^{x} and R^{y} both represent H, and L¹ and L² both represent —N(H)—S(O)₂, then Ar¹ and Ar² do not both represent 1-hydroxynaphthyl,
   which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl groups (where q is the upper limit of the range) defined herein may be straight-chain or, when there is a sufficient number (i.e. a minimum of two or three, as appropriate) of carbon atoms, be branched-chain, and/or cyclic (so forming a C_{3-q}-cycloalkyl group). Such cycloalkyl groups may be monocyclic or bicyclic and may further be bridged. Further, when there is a sufficient number (i.e. a minimum of four) of carbon atoms, such groups may also be part cyclic. Such alkyl groups may also be saturated or, when there is a sufficient number (i.e. a minimum of two) of carbon atoms, be unsaturated (forming, for example, a C_{2-q} alkenyl or a C_{2-q} alkynyl group).

The term “halo”, when used herein, includes fluoro, chloro, bromo and iodo.

Heterocycloalkyl groups that may be mentioned include non-aromatic monocyclic and bicyclic heterocycloalkyl groups (which groups may further be bridged) in which at least one (e.g. one to four) of the atoms in the ring system is other than carbon (i.e. a heteroatom), and in which the total number of atoms in the ring system is between three and twelve (e.g. between five and ten). Further, such heterocycloalkyl groups may be saturated or unsaturated containing one or more double and/or triple bonds, forming for example a C_{2-q} heterocycloalkenyl (where q is the upper limit of the range) or a C_{7-q} heterocycloalkynyl group. C_{2-q} heterocycloalkyl groups that may be mentioned include 7-azabicyclo-[2.2.1]heptanyl, 6-azabicyclo[3.1.1]heptanyl, 6-azabicyclo[3.2.1]-octanyl, 8-azabicyclo[3.2.1]octanyl, aziridinyl, azetidinyl, dihydropyranyl, dihydropyridyl, dihydropyrrolyl (including 2,5-dihydropyrrolyl), dioxolanyl (including 1,3-dioxolanyl), dioxanyl (including 1,3-dioxanyl and 1,4-dioxanyl), dithianyl (including 1,4-dithianyl), dithiolanyl (including 1,3-dithiolanyl), imidazolidinyl, imidazolinyl, morpholinyl, 7-oxabicyclo[2.2.1]heptanyl, 6-oxabicyclo[3.2.1]-octanyl, oxetanyl, oxiranyl, piperazinyl, piperidinyl, pyranyl, pyrazolidinyl, pyrrolidinonyl, pyrrolidinyl, pyrrolinyl, quinuclidinyl, sulfolanyl, 3-sulfolenyl, tetrahydropyranyl, tetrahydrofuranyl, tetrahydropyridyl (such as 1,2,3,4-tetrahydropyridyl and 1,2,3,6-tetrahydropyridyl), thietanyl, thiiranyl, thiolanyl, thiomorpholinyl, trithianyl (including 1,3,5-trithianyl), tropanyl and the like. Substituents on heterocycloalkyl groups may, where appropriate, be located on any atom in the ring system including a heteroatom. Further, in the case where the substituent is another cyclic compound, then the cyclic compound may be attached through a single atom on the heterocycloalkyl group, forming a so-called “spiro”-compound. The point of attachment of heterocycloalkyl groups may be via any atom in the ring system including (where appropriate) a heteroatom (such as a nitrogen atom), or an atom on any fused carbocyclic ring that may be present as part of the ring system. Heterocycloalkyl groups may also be in the N— or S— oxidised form.

For the avoidance of doubt, the term “bicyclic” (e.g. when employed in the context of heterocycloalkyl groups) refers to groups in which the second ring of a two-ring system is formed between two adjacent atoms of the first ring. The term “bridged” (e.g. when employed in the context of heterocycloalkyl groups) refers to monocyclic or bicyclic groups in which two non-adjacent atoms are linked by either an alkylene or heteroalkylene chain (as appropriate).

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heterocyclic groups that may be mentioned include oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl), preferably, benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazopyridyl (including imidazo[4,5-b]pyridyl, imidazo[5,4-b]pyridyl and, preferably, imidazo[1,2-a]pyridyl), indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N— or S— oxidised form.

Heteroatoms that may be mentioned include phosphorus, silicon, boron, tellurium, selenium and, preferably, oxygen, nitrogen and sulphur.

For the avoidance of doubt, in cases in which the identity of two or more substituents in a compound of the invention may be the same, the actual identities of the respective substituents are not in any way interdependent. For example, in the situation in which X¹ and X² both represent R^{3a}, i.e. a C₁₋₆ alkyl group optionally substituted as hereinbefore defined, the alkyl groups in question may be the same or different. Similarly, when groups are substituted by more than one substituent as defined herein, the identities of those individual substituents are not to be regarded as being interdependent. For example, when X¹ represents two optional substituents —R^{3a} and —C(O)R^{3b} in which R^{3b} represents R^{3a}, then the identities of the two R^{3a} groups are not to be regarded as being interdependent. Likewise, when Ar¹ represents e.g. an aryl group substituted by G¹ in addition to, for example, C₁₋₈ alkyl, which latter group is substituted by G¹, the identities of the two G¹ groups are not to be regarded as being interdependent.

For the avoidance of doubt, X¹ and X² represent between one and three optional (i.e. X¹ and X² may not be present) substituents, which may be attached to any one of the three free positions of the benzene ring to which X¹ and/or X² (as appropriate) is attached.

For the avoidance of doubt, when a term such as “R^{4a} to R⁴ⁱ” is employed herein, this will be understood by the skilled person to mean R^{4a}, R^{4b}, R^{4c}, R^{4d}, R^{4e}, R^{4f}, R^{4g}, R^{4b} and R⁴ⁱ inclusively.

For the avoidance of doubt, where it is stated herein that “any pair of R^{16a} to R^{16c} and R^{17a} to R^{17f} . . . may . . . be linked together”, we mean that any one of R^{16a}, R^{16b}, or R^{16c} may be linked with any one of R^{17a}, R^{17b}, R^{17c}, R^{17d}, R^{17e} or R^{17f} to form a ring as hereinbefore defined. For example, R^{16a} and R^{17b} (i.e. when a G¹ group is present in which G¹ represents -A¹-R^{16a}, A¹ represents —C(O)A² and A² represents —N(R^{17b})—), or R^{16c} and R^{17f}, may be linked together with the nitrogen atom to which they are necessarily attached to form a ring as hereinbefore defined.

Further compounds of the invention that may be mentioned include those in which:

when X¹ or X² represent —N(R^{3d})C(O)R^{4c}, and R^{4c} represents R^{3a}, then R^{3a} represents a linear or branched C₁₋₆ alkyl group optionally substituted by one or more substituents selected from F, Cl, —CN, —N₃, ═O, —OR^{6a}, —N(R^{6b})R^{7b}, —S(O)ₙR^{6c}, —S(O)₂N(R^{6d})R^{7d} or —OS(O)₂N(R^{6e})R^{7e};
X¹ and X² independently represent one or more optional substituents selected from halo, —R^{3a}, —CN, —C(O)R^{3b}, —C(O)OR^{3c}, —C(O)N(R^{4a})R^{5a}, —N(R^{4b})R^{5b}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3h}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4h})R^{5h} and —OS(O)₂N(R⁴ⁱ)R⁵ⁱ.

Compounds of the invention that may be mentioned include those in which:

when, for example, L¹ represents —N(R^{w})A¹⁹- or L² represents —N(R^{z})A²⁰-; A¹⁹ and A²⁰ independently represent a single bond; and/or R^{w} and R^{z} independently represent H, then:
Y¹ or Y² (as appropriate) do not represent a benzimidazolyl (such as one attached to the L¹ or L² group via the imidazolyl moiety, e.g. benzimidazol-2-yl) group;
when Y¹ or Y² represents heteroaryl, then it is preferably a monocyclic heteroaryl group or a bicyclic heteroaryl group containing 1 to 4 heteroatoms consisting of 1, 3 or 4 nitrogen heteroatoms, 1 or 2 oxygen heteroatoms and/or 1 sulfur atom, for instance, the bicyclic heteroaryl group may contain 1 nitrogen, oxygen or sulfur heteroatom (all of which are optionally substituted by one or more substituents selected from A);
when Y¹ or Y² represents a polycyclic (e.g. bicyclic) heteroaryl group, then it is preferably not attached to the L² or L³ group via a ring containing a heteroatom;
Y¹ and/or Y² (as appropriate) represent(s) aryl or a 5- or 6-membered monocyclic ring (all of which are optionally substituted by one or more substituents selected from A).

Preferred compounds of the invention include those in which R^{x}, R^{y}, R^{w} and R^{z} independently represent H, C₁₋₁₀ (e.g. C₁₋₈) alkyl (optionally substituted by one or more substituents selected from halo, —CN, —N(R^{24a})R^{25a}, —OR^{24b} or ═O).

Compounds of the invention that may be mentioned include those in which A¹⁹ represents —C(O)N(R^{w})— and A²⁰ represents —C(O)N(R^{z})—. Preferred such compounds include those in which R^{w} and R^{z} are both H (at each occurrence).

Compounds of the invention that may also be mentioned include those in which A¹⁹ and A²⁰ both represent single bonds.

Other compounds of the invention that may be mentioned include those in which:

(a) A¹⁹ does not represent —C(O)N(R^{w})— and A²⁰ does not represent —C(O)N(R^{z})— (for example, A¹⁹ does not represent —C(O)N(H)— and A²⁰ does not represent —C(O)N(R^{z})—); and
(b) A¹⁹ and A²⁰ do not both represent single bonds.

In this respect, compounds of the invention that may be mentioned include those in which:
(i) A¹⁹ represents —C(O)N(R^{w})— and A²⁰ represents —C(O)N(R^{z})—, in which at least one of R^{w} and R^{z} represents C₁₋₁₄ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d}));
(ii) A¹⁹ represents —C(O)N(R^{w})— and A²⁰ represents a single bond, —S(O)₂— or —CH₂—;
(iii) A²⁰ represents —C(O)N(R^{z})— and A¹⁹ represents a single bond, —S(O)₂— or —CH₂—;
(iv) A¹⁹ represents a single bond and A²⁰ represents —C(O)N(R^{z})—, —S(O)₂— or —CH₂—; and
(v) A²⁰ represents a single bond and A¹⁹ represents —C(O)N(R^{w})—, —S(O)₂— or —CH₂—.

Further compounds of the invention that may be mentioned include those in which:

M¹ and M² independently represent —CH₃, —CF₃ or —N(R¹⁴)R^{15a};
R^{8a} and R^{11a} independently represent H, —CH₃, —CH₂CH₃ or —CF₃.

Preferred compounds of the invention include those in which:

X¹ and X² independently represent one or more optional substituents selected from halo (e.g. chloro), R^{3a} and —OR^{3h};
X¹ and X² are the same (i.e. they are both absent or, when present, X¹ and X² represent the same substituent(s));
R^{4e} represents R^{3a};
when any of the pairs R^{4a} and R^{5a}, R^{4b} and R^{5b}, R^{5d} and R^{5d}, R^{4f} and R^{5f}, R^{4g} and R^{5g}, R^{4h} and R^{5h} or R⁴ⁱ and R⁵ⁱ are linked together, they form a 5- or 6-membered ring optionally substituted by Cl, ═O or, preferably, F or R^{3a};
R^{3c} and R^{3j} independently represent R^{3a};
R^{3a} represents C₁₋₆ (e.g. C₁₋₄) alkyl optionally substituted by one or more substituents selected from Cl, —N₃, ═O, —N(R^{6b})R^{7b} and, preferably, F and —OR^{6a};
m and n independently represent 2;
R^{6a}, R^{6b}, R^{6c}, R^{6d} and R^{6e} independently represent H or C₁₋₃ alkyl optionally substituted by one or more fluoro atoms;
R^{7b}, R^{7d} and R^{7e} independently represent H, —S(O)₂CH₃, —S(O)₂CF₃ or C₁₋₃ alkyl optionally substituted by one or more fluoro atoms, or the relevant pairs (i.e. R^{6b} and R^{7b}, R^{6d} and R^{7d} or R^{6e} and R^{7e}) are linked together as defined herein;
when R^{6b} and R^{7b}, R^{6d} and R^{7d} or R^{6e} and R^{7e} are linked together, they form a 5- or 6-membered ring, optionally substituted by F, Cl, ═O or —CH₃;
M¹ and M² independently represent —CH₃ or —CF₃;
R^{8a}, R^{9a}, R^{10a}, R^{11a}, R^{12a}, R^{13a}, R^{14a} and R^{15a} independently represent H or —CH₃;
A represents aryl (e.g. phenyl) optionally substituted by B; C₁₋₆ alkyl optionally substituted by G¹ and/or Z¹; or G¹;
G¹ represents halo, cyano, N₃, —NO₂ or -A¹-R^{16a};
A¹ represents —C(O)A², —N(R^{17a})A⁴- or —OA⁵-;
A² represents a single bond or —O—;
A⁴ represents —C(O)N(R^{17d})—, —C(O)O— or, more preferably, a single bond or —C(O)—;
A⁵ represents —C(O)— or, preferably, a single bond;
Z¹ represents ═NOR^{16b}, ═NCN or, preferably, ═O;
B represents aryl (e.g. phenyl) optionally substituted by G²; C₁₋₆ alkyl optionally substituted by G² and/or Z²; or, preferably G²,
G² represents cyano or, more preferably, halo, —NO₂ or -A⁶-R^{18a};
A⁶ represents a single bond, —N(R^{19a})A⁹- or —OA¹⁰-;
A⁹ represents —C(O)N(R^{19d})—, —C(O)O— or, more preferably, a single bond or —C(O)—;
A¹⁰ represents a single bond;
Z² represents ═NOR^{18b}, ═NCN or, more preferably, ═O;
R^{16a}, R^{16b}, R^{16c}, R^{17a}, R^{17b}, R^{17c}, R^{17d}, R^{17e}; R^{17f}, R^{18a}; R^{18b}; R^{18c}; R^{19a}, R^{19b}, R^{19c}, R^{19d}, R^{19e} and R^{19f} are independently selected from hydrogen, aryl (e.g. phenyl) or heteroaryl (which latter two groups are optionally substituted by G³) or C₁₋₆ (e.g. C₁₋₄) alkyl (optionally substituted by G³ and/or Z³), or the relevant pairs are linked together as hereinbefore defined;
when any pair of R^{16a} to R^{16c} and R^{17a} to R^{17f}, or R^{18a} to R^{18b} and R^{19a} to R^{19f} are linked together, they form a 5- or 6-membered ring, optionally substituted by one or more (e.g. one or two) substituents selected from G³ and/or Z³;
G³ represents halo or -A¹¹-R^{20a};
A¹¹ represents a single bond or —OA¹⁵-;
A¹⁵ represents a single bond;
Z³ represents ═O;
R^{20a}, R^{20b}, R^{20c}, R^{21a}, R^{21b}, R^{21c}, R^{21d}, R^{21e} and R^{21f} are independently selected from H or C₁₋₃ (e.g. C₁₋₂) alkyl (e.g. methyl) optionally substituted by one or more halo (e.g. fluoro) atoms, or the relevant pairs are linked together as defined herein;
when any pair of R^{20a} to R^{20c} and R^{21a} to R^{21f} are linked together, they form a 5- or 6-membered ring, optionally substituted by one or more (e.g. one or two) substituents selected from halo and C₁₋₂ alkyl (e.g. methyl);
R^{x}, R^{y}, R^{w} and R^{z} independently represent H or C₁₋₆ (e.g. C₁₋₃) alkyl optionally substituted as defined herein, for example by one or more halo (e.g. fluoro) groups;
R^{x} and R^{y} are the same;
R^{w} and R^{z} are the same;
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} independently represent hydrogen or C₁₋₂ alkyl optionally substituted by ═O or, more preferably, one or more fluoro atoms.

Preferred aryl and heteroaryl groups that Ar¹ and Ar² may represent include optionally substituted (i.e. by A) phenyl, naphthyl, pyrrolyl, furanyl, thienyl (e.g. thien-2-yl or thien-3-yl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred values include optionally substituted thienyl, thiazolyl or pyridyl or, more preferably, optionally substituted naphthyl (e.g. 1-naphthyl or 2-naphthyl) or phenyl.

Preferred substituents on Ar¹ and Ar² groups include:

halo (e.g. fluoro, chloro or bromo);
cyano;

### —NO₂;

C₁₋₆ alkyl, which alkyl group may be cyclic (e.g. cyclohexyl), part-cyclic, unsaturated or, preferably, linear or branched (e.g. C₁₋₄ alkyl (such as ethyl, n-propyl, isopropyl, n-butyl, t-butyl or, preferably, methyl), all of which are optionally substituted with one or more halo (e.g. fluoro) groups (so forming, for example, fluoromethyl, difluoromethyl or, preferably, trifluoromethyl);
heterocycloalkyl, such as a 5- or 6-membered heterocycloalkyl group, preferably containing a nitrogen atom and, optionally, a further nitrogen or oxygen atom, so forming for example morpholinyl (e.g. 4-morpholinyl), piperazinyl (e.g. 4-piperazinyl) or piperidinyl (e.g. 1-piperidinyl and 4-piperidinyl) or pyrrolidinyl (e.g. 1-pyrrolidinyl), which heterocycloalkyl group is optionally substituted by one or more (e.g. one or two) substituents selected from C₁₋₃ alkyl (e.g. methyl) and ═O;

### —OR²⁶.

### —C(O)OR²⁶:

### —C(O)R²⁶; and

### —N(R²⁶)R²⁷;

wherein R²⁶ and R²⁷ independently represent, on each occasion when mentioned above, aryl (e.g. phenyl) optionally substituted by one or more halo or C₁₋₃ (e.g. C₁₋₂) alkyl groups (which alkyl group is optionally substituted by one or more halo (e.g. fluoro) atoms) or, more preferably, H or C₁₋₆ alkyl, such as C₁₋₄ alkyl (e.g. ethyl, n-propyl, n-butyl, t-butyl or, preferably, methyl or isopropyl) optionally substituted by one or more halo (e.g. fluoro) groups (so forming e.g. a trifluoromethyl group).

Preferred compounds of the invention (particularly in those compounds in which A¹⁹ and A²⁰ both represent single bonds) include those in which:

Y¹ and Y² independently (i.e. either one or, more preferably, both) do not represent H;
when Ar¹ and Ar² are substituted, they are preferably substituted by one to three (e.g. one or two) substituents as defined herein;
A represents G¹ or C₁₋₄ alkyl (e.g. t-butyl or methyl) optionally substituted by one or more G¹ groups (e.g. by halo, such as fluoro);
G¹ represents halo (e.g. F or Cl) or -A¹-R^{16a};
A¹ represents a single bond or, more preferably, —C(O)A²- or —OA⁵-;
A² represents —O—;
A⁵ represents a single bond;
R^{16a} represents aryl (e.g. phenyl) or heteroaryl, which are both optionally substituted by one or more G³ groups or, more preferably, H or C₁₋₆ alkyl (e.g. cyclohexyl or, more preferably, C₁₋₂ alkyl) optionally substituted by one or more G³ (e.g. fluoro) substituents;
G³ represents fluoro or -A¹¹-R^{20a};
X¹ and X² independently represent halo (e.g. chloro) or is/are not present; when X¹ and X² is/are present, they independently represent two or, preferably, one substituent as defined herein;
when X¹ and X² are present, they represent one substituent (as defined herein) preferably attached to the position α to the —N(R^{z})—Y¹ or —N(R^{w})—Y² substituent in the compound of formula I;
Ar¹ and Ar² independently represent phenyl or naphthyl optionally substituted as defined herein;
when Ar¹ and Ar² represent a phenyl group, it may be unsubstituted or substituted with one substituent or with two substituents (so forming, for example a 2,3-, a 3,5-, a 3,4- or a 2,4-substitution pattern) as defined herein;
when Ar¹ or Ar² represents naphthyl, it is preferably unsubstituted;
Ar¹ and Ar² are the same;
R^{x} and R^{y} independently represent H;
R^{w} and R^{z} independently represent H or C₁₋₃ (e.g. C₁₋₂) alkyl (e.g. n-propyl or, more preferably, methyl), which group may be optionally substituted by a phenyl group, optionally substituted by C₁₋₃ alkyl, such as methyl.

Particularly preferred substituents on Ar¹ and Ar² groups (particularly in those compounds in which A¹⁹ and A²⁰ represent single bonds) include, for example, one or more substituents selected from cyclohexyl or, more preferably, halo (e.g. chloro, fluoro or bromo), —C(O)OH, —CH₃, —CF₃, t-butyl, —OCH₃, —OCF₃ or —O-isopropyl substituents.

In addition to the above-mentioned preferences, preferred compounds of the invention (particularly in those compounds in which A¹⁹ and A²⁰ both represent —C(O)N(H)—, or one of A¹⁹ or A²⁰ represents —C(O)N(H)— and the other represents a single bond and Y¹ and/or Y² (as appropriate) represents H) may also include those in which:

when Ar¹ and Ar² are substituted, they are preferably substituted by one to three (e.g. two or, more preferably, one) substituents as defined herein;
A represents G¹ or C₁₋₃ alkyl (e.g. methyl) optionally substituted by one or more
G¹ groups (e.g. halo, such as fluoro);
G¹ represents halo (e.g. fluoro or chloro), cyano, —NO₂ or -A¹-R^{16a};
A¹ represents —C(O)A²- or —OA⁵-;
A² and A⁵ independently represent a single bond;
R^{16a} represents C₁₋₄ (e.g. C₁₋₂) alkyl (e.g. n-butyl or methyl) or an aryl (e.g. phenyl) or heteroaryl group, which latter two are optionally substituted by one or more G³ groups;
Ar¹ and Ar² independently represent phenyl optionally substituted as defined herein.

Particularly preferred substituents on Ar¹ and Ar² groups (particularly in those compounds those in which A¹⁹ and A²⁰ both represent —C(O)N(H)—, or one of A¹⁹ or A²⁰ represents —C(O)N(H)— and the other represents a single bond and Y¹ and/or Y² (as appropriate) represents H) include, for example, one or more substituents selected from halo (e.g. chloro, fluoro or bromo), cyano, —C(O)CH₃, —CH₃, —CF₃, —NO₂, —OCH₃, —O-n-butyl and —O-phenyl (i.e. phenoxy).

Preferred compounds of the invention include those in which A¹⁹ and/or A²⁰ represent —CH₂— or, more preferably, a single bond, or —C(O)N(R^{w})— or —C(O)N(R^{z})— (as appropriate).

Compounds of the invention that may be mentioned include those in which A¹⁹ and A²⁰ are both the same. Other compounds of the invention that may be mentioned include those in which one of A¹⁹ or A²⁰ represents —S(O)₂— and the other represents —C(O)N(R^{w})— or —C(O)N(R^{z})— (as appropriate).

Compounds of the invention that may be mentioned (particularly in those compounds in which A¹⁹ and A²⁰ both represent —S(O)₂—) also include those in which:

when Ar¹ and Ar² are substituted, they are preferably substituted by one to two substituents as defined herein;
A represents G¹ or C₁₋₄ alkyl (preferably n-butyl or methyl), optionally substituted by one or more (e.g. three) G¹ groups (such as halo, e.g. fluoro));
G¹ represents halo (e.g. fluoro or chloro), cyano, —NO₂ or -A¹-R^{16a};
A¹ represents a single bond, —N(R^{17a})A⁴, —C(O)A²- or —O-A⁵-;
A² represents —O—;
A⁴ represents —C(O)— or a single bond;
A⁵ represents a single bond;
R^{16a} represents H or C₁₋₄ alkyl (e.g. C₁₋₂ alkyl), optionally substituted by one or more (e.g. three) G³ (e.g. fluoro) substituents);
   R^{17a} represents H;
   Ar¹ and Ar² independently represent phenyl or naphthyl optionally substituted as defined herein (the latter being preferably unsubstituted).

Particularly preferred substituents on Ar¹ and Ar² groups (particularly in those compounds in which A¹⁹ and A²⁰ represent —S(O)₂—) include, for example, one or more substituents selected from halo (e.g. chloro or fluoro), cyano, —NO₂, —C(O)OH, —CF₃, —OCH₃, —OCF₃, —NH₂, —N(H)—C(O)CH₃, -n-butyl and —O-n-butyl.

Compounds of the invention that may be mentioned (particularly in those compounds in which A¹⁹ and A²⁰ both represent —CH₂—) may also include those in which:

when Ar¹ and Ar² are substituted, they are preferably substituted by one to two substituents as defined herein;
A represents G¹ or C₁₋₄ alkyl (preferably methyl, which is optionally substituted by one or more (e.g. three) G¹ groups (such halo, e.g. fluoro));
G¹ represents halo (e.g. fluoro or chloro) or -A¹-R^{16a},
A¹ represents —O-A⁵-;
A⁵ represents a single bond;
R^{16a} represents C₁₋₄ alkyl (preferably C₁₋₂ alkyl), optionally substituted by one or more (e.g. three) G³ groups (e.g. fluoro or -A¹¹-R^{20a});
A¹¹ represents a single bond;
R^{20a} represents and aryl (e.g. phenyl);
Ar¹ and Ar² independently represent phenyl optionally substituted as defined herein.

Particularly preferred substituents on Ar¹ and Ar² groups (particularly in those compounds in which A¹⁹ and A²⁰ represent —S(O)₂—) include, for example, one or more substituents selected from halo (e.g. chloro or fluoro), —CF₃, —OCH₃, —CH₃ and —O—CH₂-phenyl.

Compounds of the invention that may be mentioned (particularly in those compounds in which one of A¹⁹ or A²⁰ represents —S(O)₂— and the other represents —C(O)N(H)—) may also include those in which:

when Ar¹ and Ar² are substituted, they are preferably substituted by one to two substituents as defined herein;
A represents G¹;
G¹ represents halo (e.g. fluoro or chloro) or —NO₂;
Ar¹ and Ar² independently represent phenyl optionally substituted as defined herein.

Particularly preferred substituents on Ar¹ and Ar² groups (particularly in those compounds in which one of A¹⁹ or A²⁰ represents —S(O)₂— and the other represents —C(O)N(H)—) include, for example, one or more substituents selected from halo (e.g. chloro) and —NO₂.

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) for compounds of formula I in which A¹⁹ and A²⁰ represent a single bond, and in particular for the preparation of compounds of formula I in which R^{x} and R^{y} do not represent hydrogen, reaction of a compound of formula II,

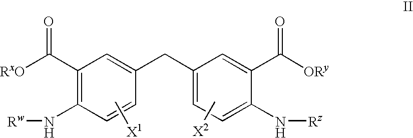

or a protected (e.g. at one of the amino groups) derivative thereof, wherein R^{x} and R^{y} are as hereinbefore defined but preferably do not represent hydrogen, and) X¹, X², R^{w} and R^{z} are as hereinbefore defined, with a compound of formula III,

Ar^{a}-L^{a} III

wherein Ar^{a} represents Ar¹ or Ar² (as appropriate/required) and L^{a} represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂ and Ar¹ and Ar² are as hereinbefore defined, for example optionally in the presence of an appropriate metal catalyst (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the reagent may itself act as a solvent (e.g. when Ar^{a} represents phenyl and L^{a} represents bromo, i.e. bromobenzene). This reaction may be carried out at room temperature or above (e.g. at a high temperature, such as the reflux temperature of the solvent system that is employed) or using microwave irradiation. The skilled person will appreciate that this preparation may result in compounds of formula I in which one of Y¹ and Y² represents H, or neither of Y¹ or Y² represent H. Hence, the desired products may be separated in accordance with standard techniques. Compounds in which one of Y¹ or Y² represent H may be prepared in higher yields by either employing less than two equivalents of a compound of formula III in the reaction mixture, or by employing a mono-protected (at a single amino group) compound of formula II. Compounds of formula I in which neither Y¹ or Y² represent H may be prepared in higher yield by employing an excess (i.e. more than two equivalents) of (a) compound(s) of formula III in the reaction mixture;
(ii) for compounds of formula I in which R^{w} and/or R^{z} do not represent hydrogen, reaction of a corresponding compound of formula I in which R^{z} and/or R^{z} (as appropriate) do represent hydrogen with a compound of formula IV,

R^{wz}-L^{b} IV

wherein R^{wz} represents either R^{w} or R^{z} (as appropriate) as hereinbefore defined provided that it/they does/do not represent hydrogen, and L^{b} represents a suitable leaving group such as one hereinbefore defined in respect of L^{a} or —Sn(alkyl)₃ (e.g. —SnMe₃ or —SnBu₃), or a similar group known to the skilled person, under reaction conditions known to those skilled in the art, for example such as those described in respect of process step (i) above or, for example in the case where L^{b} represents a leaving group such as iodo, bromo, chloro or a sulfonate group, the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine). In the case when L^{b} represents —B(OH)₂ or —Sn(alkyl)₃, the reaction may be performed in the presence of a suitable catalyst system, e.g. a metal (or a salt or complex thereof) such as CuI, Pd/C, PdCl₂, Pd(OAc)₂, Pd(Ph₃P)₂Cl₂, Pd(Ph₃P)₄, Pd₂(dba)₃ or NiCl₂ and a ligand such as t-Bu₃P, (C₆H₁₁)₃P, Ph₃P, AsPh₃, P(o-Tol)₃, 1,2-bis(diphenylphosphino)ethane, 2,2′-bis(di-tert-butylphosphino)-1,1′-bi-phenyl, 2,2′-bis(diphenylphosphino)-1,1′-bi-naphthyl, 1,1′-bis(diphenylphosphinoferrocene), 1,3-bis(diphenylphosphino)-propane, xantphos, or a mixture thereof, together with a suitable base such as, Na₂CO₃, K₃PO₄, Cs₂CO₃, NaOH, KOH, K₂CO₃, CsF, Et₃N, (i-Pr)₂NEt, t-BuONa or t-BuOK (or mixtures thereof) in a suitable solvent such as dioxane, toluene, ethanol, dimethylformamide, ethylene glycol dimethyl ether, water, dimethylsulfoxide, acetonitrile, dimethylacetamide, N-methylpyrrolidinone, tetrahydrofuran or mixtures thereof. Further, the skilled person will appreciate that R^{wz} may be contain a double bond, for example α to the L^{b} substituent (which double bond may migrate after reaction). In this instance, it may be desired to subsequently reduce the double bond to provide a saturated R^{wz} group, for example as described hereinafter;
(iii) for compounds of formula I that contain only saturated alkyl groups (for example, when R^{w} and/or R^{z} represent optionally substituted saturated C₁₋₁₄ alkyl), reduction of a corresponding compound of formula I that contains an unsaturation, such as a double or triple bond (e.g. for compounds of formula I in which R^{w} and/or R^{z} represent C₂₋₁₄ alkenyl), in the presence of suitable reducing conditions, for example by catalytic (e.g. employing Pd) hydrogenation;
(iv) for compounds of formula I that contain amine groups (for example, where G¹ represents —NH₂, or, for compounds of formula I in which either -L¹-Y¹ or -L²-Y² represents —NH₂) reduction of a corresponding compound of formula I that contains a group that may be reduced to an amine group, such a nitro or azide group (e.g. G¹ represents —NO₂ or —N₃), in the presence of suitable reducing conditions, for example by catalytic (e.g. employing Pd) hydrogenation or employing an appropriate reducing agent (such as trialkylsilane, e.g. triethylsilane). The skilled person will also appreciate that the amine, once formed, may further be substituted (e.g. alkylated) using any appropriate process, for example those described herein;
(v) for compounds of formula I in which A¹⁹ and A²⁰ independently represent a single bond or —CH₂—, and R^{w} and/or R^{z} represents optionally substituted C₂₋₁₄ alkyl, reductive amination of a compound of formula II as defined above, with a compound of formula V,

R^{wz1}═O V

wherein R^{wz1} represents C₁₋₁₃ alkyl optionally substituted with the substituents hereinbefore defined in respect of R^{w} and/or R^{z} (and the compound of formula V is thus either an aldehyde or ketone). Reductive amination (which comprises condensation followed by reduction) reaction conditions are well known to those skilled in the art, for example, such reactions may be performed in the presence of a suitable chemoselective reducing agent, such as sodium cyanoborohydride, sodium triacetoxyborohydride or borane (or various complexes thereof). Alternatively, the reduction step may be performed as a completely separate step after the condensation step (which condensation step may itself be promoted when performed in the presence of a suitable reagent such as a titanium based reagent, e.g. Ti(Oi-Pr)₄), in the presence of a stronger reducing agent such as sodium borohydride or borane (and various complexes thereof);
(vi) reaction of a compound of formula VI,

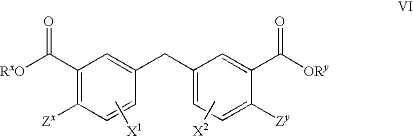

wherein Z^{x} and Z^{y} independently represent a suitable leaving group such as chloro, bromo, iodo, a sulfonate group (e.g. —OS(O)₂CF₃, —OS(O)₂CH₃, —OS(O)₂PhMe or a nonaflate), —B(OH)₂, —B(ORⁿ)₂, —Sn(Rⁿ)₃ or diazonium salts, in which each Rⁿ independently represents a C₁₋₆ alkyl group, and R^{x}, R^{y}, X¹ and X² are as hereinbefore defined, with a compound of formula VII,

Y^{a}-A²¹-NH₂ VII

wherein Y^{a} is as hereinbefore defined, A²¹ represents A¹⁹ or A²⁰ (as required/appropriate) (where A¹⁹ or A²⁰ preferably independently represent —N(H)—C(O)—N(H)— or a single bond) under suitable reaction conditions known to those skilled in the art, such as those described hereinbefore in respect of process step (i);
(vii) for compounds of formula I in which A¹⁹ and/or A²⁰ represents —CH₂—, reductive amination of a compound of formula II as defined above, in the presence of a compound of formula VIII,

Ar^{a}CH═O VIII

wherein Ar^{a} is as hereinbefore defined. Reductive amination (which comprises condensation followed by reduction) reaction conditions are well known to those skilled in the art, for example, those described hereinbefore in respect of process step (v);
(viii) for compounds of formula I in which A¹⁹ and/or A²⁰ represents —CH₂—, reaction of a compound of formula II as defined above, with a compound of formula IX,

Ar^{a}C(O)Cl IX

wherein Ar^{a} is as hereinbefore defined, under conditions known to one skilled in the art, for example, those described hereinbefore in respect of process step (ii), followed by reduction of the resulting compound (either in a separate reaction or in one pot), under conditions known to one skilled in the art, for example using a suitable reducing agent, such as borane (and various complexes thereof);
(ix) for compounds of formula I in which R^{x} and R^{y} represent hydrogen, hydrolysis of a corresponding compound of formula in which R^{x} and R^{y} do not represent hydrogen, or other carboxylic acid or ester protected derivatives (e.g. amide derivatives) thereof, under standard conditions, for example in the presence of an aqueous solution of base (e.g. aqueous 2M NaOH) optionally in the presence of an (additional) organic solvent (such as dioxane, diethyl ether or MeOH), which reaction mixture may be stirred at room or, preferably, elevated temperature (e.g. about 120° C.) for a period of time until hydrolysis is complete (e.g. 5 hours);
(x) for compounds of formula I in which R^{x} and R^{y} do not represent hydrogen (and are preferably the same), esterification of corresponding compounds of formula I in which R^{x} and R^{y} represent hydrogen (or trans-esterification of compounds of formula I in which R^{x} and R^{y} do not represent hydrogen or the same value of the corresponding R^{x} and R^{y} groups in the compound of formula I to be prepared), in the presence of a compound of formula X,

R^{b}—OH X

wherein R^{b} represents R^{x} or R^{y} (as appropriate/required) provided that it does not represent hydrogen, under standard conditions, for example in the presence of acid (e.g. concentrated H₂SO₄) at elevated temperature, such as at the reflux temperature of the alcohol of formula X;
(xi) for compounds of formula I in which A¹⁹ and A²⁰ represent —S(O)₂— or —CH₂—, reaction of a compound of formula II with a compound of formula XI,

Y^{a}-A^{x}-L^{c} XI

wherein Y^{a} represents Ar¹ or Ar² (as appropriate/required) as hereinbefore defined and L^{c} represents a suitable leaving group, for example, fluoro (especially when A^{x} represents —S(O)₂—) or a suitable leaving group such as one defined hereinbefore in respect of L^{a}, and A^{x} represents either —CH₂— or —S(O)₂—, under suitable conditions as known to one skilled in the art, for example the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine). Alternatively, reaction conditions such as those described hereinbefore in respect of process step (ii) may be employed. The skilled person will appreciate that for the preparation of compounds of formula I in where either A¹⁹ represents a single bond and Y¹ represents H, or A²⁰ represents a single bond and Y² represents H, then a mono-protected (at a single amino group) compound of formula II may be employed or the reaction may be performed with less than 2 equivalents of the compound of formula XI. The skilled person will also appreciate that for preparation of compounds of formula I in which A¹⁹ and A²⁰, and/or, Ar¹ and Ar² are different, two different compounds of formula XI will need to be employed in successive reaction steps;
(xii) for compounds of formula I in which A¹⁹ and A²⁰ both represent —C(O)N(H)—, reaction of a compound of formula II, or a protected (e.g. at one of the amino groups) derivative thereof, with either:
(A) a compound of formula XII,

Y^{a}—N═C═O XII;

or
(B) with CO (or a reagent that is a suitable source of CO (e.g. Mo(CO)₆ or CO₂(CO)₈)) in the presence of a compound of formula XIII,

Y^{a}—NH₂ XIII

wherein, in both cases, Y^{a} represents Ar¹ or Ar² (as appropriate/required) as hereinbefore defined. For example, in the case of (A) above, in the presence of a suitable solvent (e.g. THF, dioxane or diethyl ether) under reaction conditions known to those skilled in the art (e.g. at room temperature). In the case of (B), suitable conditions will be known to the skilled person, for example the reactions may be carried out in the presence of an appropriate catalyst system (e.g. a palladium catalyst), preferably under pressure and/or under microwave irradiation conditions. The skilled person will appreciate that the compound so formed may be isolated by precipitation or crystallisation (from e.g. n-hexane) and purified by recrystallisation techniques (e.g. from a suitable solvent such as THF, hexane (e.g. n-hexane), methanol, dioxane, water, or mixtures thereof). The skilled person will appreciate that for the preparation of compounds of formula I in which one of Y¹ and Y² represents H, then a mono-protected (at a single amino group) compound of formula II may be employed or the reaction may be performed with less than 2 equivalents of the compound of formula XII or XIII (as appropriate). The skilled person will also appreciate that for preparation of compounds of formula I in which L¹-Y¹ represents —N(R^{w})—C(O)N(R^{w})—Ar¹ and L²-Y² represents —N(R^{z})—C(O)N(R^{z})—Ar², and L¹-Y¹ and L²-Y² are different, two different compounds of formula XII or XIII (as appropriate) will need to be employed in successive reaction steps;
(xiii) for compounds of formula I in which A¹⁹ and A²⁰ both represent —C(O)N(H)—, reaction of a compound of formula XIV,

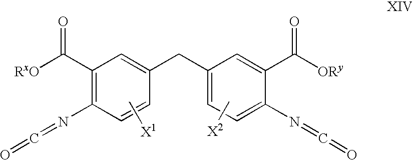

wherein R^{x}, R^{y}, X¹ and X² are as hereinbefore defined, with a compound of formula XIII as defined above, under reaction conditions known to those skilled in the art, such as those described hereinbefore in respect of process step (xii); or
(xiv) particularly for compounds of formula I in which R^{x} and R^{y}, X¹ and X² and Y¹ and Y² are the same, reaction of a compound of formula XV (or two different compounds of formula XV for preparation of compounds of formula I in which R^{x} and R^{y}, X¹ and X² and/or Y¹ and Y² are different),

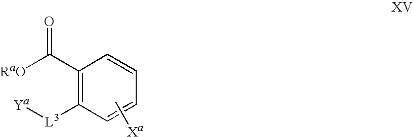

wherein R^{a} represents R^{x} or R^{y} (as required/appropriate and in which these substituents are preferably other than hydrogen and are preferably the same), L³ represents L¹ or L² (as required/appropriate and in which these substituents are preferably the same), X^{a} represents X¹ or X² (as required/appropriate and in which these substituents are preferably the same), Y^{a} is as hereinbefore defined, with formaldehyde (e.g. in the form of paraformaldehyde or an aqueous solution of formaldehyde such as a 3% aqueous solution), for example under acidic conditions (e.g. in the presence of aqueous HCl) at or above room temperature (e.g. at between 50° C. and 70° C.). Preferably, the formaldehyde is added (e.g. slowly) to an acidic solution of the compound of formula XV at about 50° C., with the reaction temperature rising to about 70° C. after addition is complete. When acidic conditions are employed, precipitation of the compound of formula I may be effected by the neutralisation (for example by the addition of a base such as ammonia).

Compounds of formula II (or protected, e.g. mono-protected derivatives thereof) may be prepared by reduction of a compound of formula XVI,

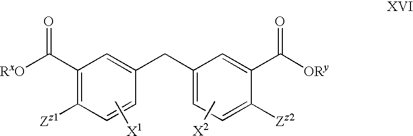

wherein Z^{z1} and Z^{z2} independently represent —N₃, —NO₂ or one of Z^{z1} or Z^{z2} may represent a protected —NH₂ group (for instance, in the case where appropriate mono-protected derivatives of compounds of formula II are required) under standard reaction conditions known to those skilled in the art, in the presence of a suitable reducing agent, for example reduction by catalytic hydrogenation (e.g. in the presence of a palladium catalyst in a source of hydrogen) or employing an appropriate reducing agent (such as trialkylsilane, e.g. triethylsilane).

Compounds of formula II (or protected derivatives thereof) may also be prepared by reaction of a compound of formula VI as defined above, with ammonia, or preferably with a protected derivative thereof (e.g. benzylamine or Ph₂C═NH), under conditions such as those described hereinbefore in respect of preparation of compounds of formula I (process step (vi) above).

Compounds of formula II or compounds of formula VI may be prepared by:

(I) reaction of a compound of formula XVII,

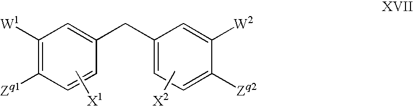

wherein Z^{q1} and Z^{q2} respectively represent Z^{x} and Z^{y} (in the case of preparation of compounds of formula VI) or —NH₂ (or preferably a protected derivative thereof; in the case of preparation of compounds of formula II), one of W¹ and W² represents hydrogen and the other represents hydrogen or —C(O)OR^{x} or —C(O)OR^{y} (as appropriate), and X¹, X², R^{x}, R^{y}, Z^{x} and Z^{y} are as hereinbefore defined, with a suitable reagent such as phosgene or triphosgene in the presence of an appropriate base (e.g. triethylamine), followed by reaction in the presence of a compound of formula XVIII,

HO—R^{xy} XVIII

wherein R^{xy} represents R^{x} or R^{y} (as appropriate), hence undergoing a hydrolysis or alcoholysis reaction step;

(II) for compounds of formula II or VI in which R^{x} and/or R^{y} represent hydrogen, formylation of a compound of formula XVII as hereinbefore defined, for example in the presence of suitable reagents such as P(O)Cl₃ and DMF, followed by oxidation under standard conditions;

(III) reaction of a compound of formula XIX,

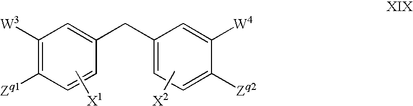

wherein one of W³ and W⁴ represents a suitable leaving group such as one defined by Z^{x} and Z^{y} above and the other also represents such a leaving group or —C(O)OR^{x} or —C(O)OR^{y} (as appropriate), and X¹, X², R^{x}, R^{y}, Z^{q1} and Z^{q2} are as hereinbefore defined, with CO (or a reagent that is a suitable source of CO (e.g. Mo(CO)₆ or CO₂(CO)₈)) followed by reaction in the presence of a compound of formula XVIII as hereinbefore defined, under reaction conditions known to those skilled in the art, for example such as those hereinbefore described in respect of preparation of compounds of formula I (process step (xii)(B) above), e.g. the carbonylation step being performed in the presence of an appropriate precious metal (e.g. palladium) catalyst;

(IV) reaction of a compound of formula XX,

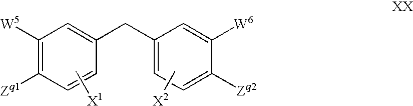

wherein one of W⁵ and W⁶ represents a suitable group such as an appropriate alkali metal group (e.g. sodium, potassium or, especially, lithium), a —Mg-halide or a zinc-based group, the other one of W⁵ or W⁶ may also represent such as group or may represent —C(O)OR^{x} or —C(O)OR^{y} (as appropriate), and X¹, X², R^{x} and R^{y} are as hereinbefore defined, with e.g. CO₂ (in the case where R^{x} and/or R^{y} in the compound of formula II or VI to be prepared represents hydrogen) or a compound of formula XXI,

L^{z}-C(O)OR^{xy} XXI

wherein L^{z} represents a suitable leaving group, such as chloro or bromo or a C₁₋₁₄ (such as C₁₋₆ (e.g. C₁₋₃) alkoxy group), and R^{xy} is as hereinbefore defined, provided that it does not represent hydrogen, under reaction conditions known to those skilled in the art. The skilled person will appreciate that this reaction step may be performed directly after (i.e. in the same reaction pot) the preparation of compounds of formula XX.

Compounds of formula VI in which Z^{x} and Z^{y} both represent a sulfonate group may be prepared by reaction of a compound of formula XXII,

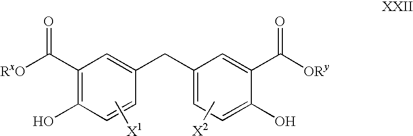

wherein R^{x} and R^{y} are as hereinbefore defined (and preferably do not represent hydrogen) and X¹ and X² are as hereinbefore defined, with an appropriate reagent for the conversion of the hydroxyl group to the sulfonate group (e.g. tosyl chloride, mesyl chloride, triflic anhydride and the like) under conditions known to those skilled in the art, for example in the presence of a suitable base and solvent (such as those described above in respect of process step (i), e.g. an aqueous solution of K₃PO₄ in toluene) preferably at or below room temperature (e.g. at about 10° C.).

Compounds of formula XIV may be prepared by reaction of a corresponding compound of formula II in which R^{z} and R^{w} represent hydrogen, with phosgene or triphosgene, for example in the presence of a suitable base (e.g. one hereinbefore defined in respect of preparation of compounds of formula I (process step (i), e.g. triethylamine). When the compound of formula XIV is synthesised accordingly, it need not be isolated and/or purified when further employed in the synthesis of a compound of formula I (see process step (xiii) above).

Compounds of formula XV in which L³ is as hereinbefore defined (as required/appropriate) may be prepared by reduction of a compound of formula XXIII,

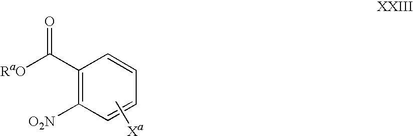

so forming a corresponding compound of formula XXIIIA,

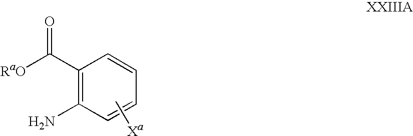

wherein R^{a} and X^{a} are as hereinbefore defined, followed by reaction in the presence of a compound of formula III, XI or XII (as appropriate) as hereinbefore defined, under reaction conditions such as those described herein.

Compounds of formula XXIII may be prepared by hydrolysis of a compound of formula XXIV,

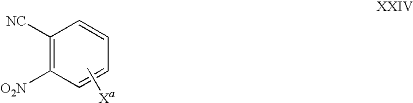

wherein X^{a} is as hereinbefore defined, followed by, if necessary, alcoholysis in the presence of a compound of formula XI in which R^{xy} does not represent hydrogen.

Compounds of formula XVII or XIX may be prepared by reduction of a compound of formula XXV,

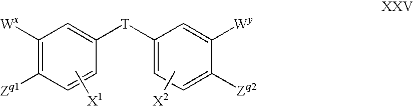

wherein W^{x} represents W¹ or W³ (as appropriate), W^{y} represents W² or W⁴ (as appropriate), T represents —C(O)— or —CH(OH)— and W¹, W², W³, W⁴, X¹, X², Z^{q1} and Z^{q2} are as hereinbefore defined, under standard reaction conditions known to those skilled in the art, for example reduction in the presence of a suitable reducing reagent such as LiAlH₄, NaBH₄ or trialkylsilane (e.g. triethylsilane) or reduction by hydrogenation (e.g. in the presence of Pd/C).

Alternatively, compounds of formula XVII or XIX may be prepared by reaction of a compound of formula XXVI,

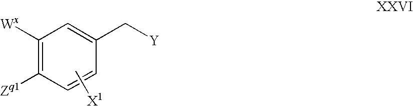

wherein Y represents a suitable group such as —OH, bromo, chloro or iodo, and W^{x}, Z^{q1} and X¹ are as hereinbefore defined, with a compound of formula XXVIII as defined hereinafter in which M represents hydrogen under standard conditions, for example, such as those described hereinafter in respect of preparation of compounds of formula XXV in which T represents —C(O)— (e.g. in the presence of a Lewis or Brønsted acid). Alternatively, such compounds may be prepared from reaction of a compound of formula XXVI in which Y represents bromo or chloro with a compound corresponding to a compound of formula XXVIII but in which M represents —BF₃K (or the like), for example in accordance with the procedures described in Molander et al, J. Org. Chem. 71, 9198 (2006).

Compounds of formula XX may be prepared in several ways. For example, compounds of formula XX in which W⁵ and/or W⁶ represent an alkali metal such as lithium, may be prepared from a corresponding compound of formula XVII (in particular those in which Z^{q1} and/or Z^{q2} represents a chloro or sulfonate group or, especially, a protected —NH₂ group, wherein the protecting group is preferably a lithiation-directing group, e.g. an amido group, such as a pivaloylamido group), by reaction with an organolithium base, such as n-BuLi, s-BuLi, t-BuLi, lithium diisopropylamide or lithium 2,2,6,6-tetramethylpiperidine (which organolithium base is optionally in the presence of a suitable additive, solvent or co-solvent (for example, a lithium co-ordinating agent or a polar aprotic solvent, such as an ether (e.g. dimethoxyethane, tetrahydrofuran or diethyl ether) or an amine (e.g. tetramethylethylenediamine (TMEDA), (−)sparteine or 1,3-dimethyl-3,4,5,6-tetrahydro-2(1H)-pyrimidinone (DMPU)) and the like), at sub-ambient temperatures (e.g. 0° C. to −78° C.) under an inert atmosphere. Alternatively, such compounds of formula XX may be prepared by reaction of a compound of formula XIX in which W³ and/or W⁴ represents chloro, bromo or iodo by a halogen-lithium reaction in the presence of an organolithium base such as t- or n-butyllithium under reaction conditions such as those described above. Compounds of formula XX in which W⁵ and/or W⁶ represent —Mg-halide may be prepared from a corresponding compound of formula XIX in which W³ and/or W⁴ represents halo (e.g. bromo), for example optionally in the presence of a catalyst (e.g. FeCl₃) under standard Grignard conditions known to those skilled in the art. The skilled person will also appreciate that the magnesium of the Grignard reagent or the lithium of the lithiated species may be exchanged to a different metal (i.e. a transmetallation reaction may be performed), for example to form compounds of formula XX in which W⁵ and/or W⁶ represent a zinc-based group (e.g. using ZnCl₂).

Compounds of formulae II, VI, XVI and XXII in which R^{x} and R^{y} represent hydrogen may be prepared from corresponding compounds of formula I in which R^{x} and R^{y} do not represent hydrogen under standard hydrolysis conditions, for example such as those described hereinbefore in respect of preparation of compounds of formula I (process (ix) above).

Compounds of formulae II, VI, XVI and XXII in which R^{x} and R^{y} do not represent hydrogen may be prepared from corresponding compounds of formula I in which R^{x} and R^{y} represent hydrogen under standard esterification conditions, for example such as those described hereinbefore in respect of preparation of compounds of formula I (process (x) above) in the presence of an alcohol of formula X as hereinbefore defined.

Compounds of formula XXIIIA, or protected derivatives thereof, may be prepared by reaction of a compound of formula XXVIA,

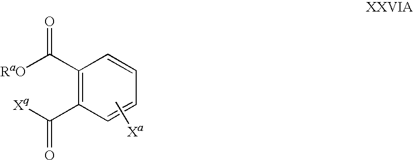

wherein X^{q} represents —OH, —NH₂ or —N₃, and R^{a} is as hereinbefore defined, under standard reaction conditions, for example:

(i) when X^{q} represents —OH, under Schmidt reaction conditions, or variants thereof, in the presence of HN₃ (which may be formed in by contacting NaN₃ with a strong acid such as H₂SO₄). Variants include reaction with diphenyl phosphoryl azide ((PhO)₂P(O)N₃) in the presence of an alcohol (such as tert-butanol; thereby forming a t-Boc protected derivative of formula XXVIA) which may result in the formation of a carbamate intermediate;

(ii) when X^{q} represents —NH₂, under Hoffmann rearrangement reaction conditions, for example in the presence of NaOBr (which may be formed by contacting NaOH and Br₂) which may result in the formation of a carbamate intermediate;

(iii) when X^{q} represents —N₃ (which compound itself may be prepared from the corresponding acyl hydrazide under standard diazotization reaction conditions, e.g. in the presence of NaNO₂ and a strong acid such as H₂SO₄ or HCl), under Curtius rearrangement reaction conditions, which may result in the formation of an intermediate isocyanate (or a carbamate if treated with an alcohol),

all of which may be followed by, if necessary (e.g. if the formation of the free amine is desired), hydrolysis, for example in the presence of water and base (e.g. one hereinbefore described in respect of process step (i) above) when a lower alkyl carbamate (e.g. methyl or ethyl carbamate) is formed as an intermediate or under acidic conditions when e.g. a tert-butyl carbamate is formed as an intermediate, or, when a benzyl carbamate intermediate is formed, under hydrogenation reaction conditions (e.g. catalytic hydrogenation reaction conditions in the presence of a precious metal catalyst such as Pd).

Alternatively, compounds of formula XXIIIA in which R^{a} represents hydrogen, may be prepared by reaction of a compound of formula XXVIB,

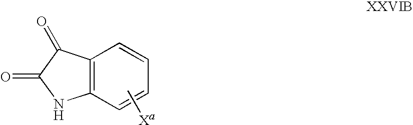

wherein X^{a} is as hereinbefore defined, under oxidation reaction conditions, for example such as those described in Sheibley, F. E. and McNulty, J. S. J. Org. Chem., 1956; 21, 171-173, e.g. in the presence of H₂O₂, which is preferably in the presence of an alkaline solution.

Compounds of formula XXV in which T represents —C(O)— may be prepared by reaction of a compound of formula XXVII,

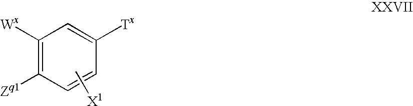

wherein T^{x} represents —C(O)Cl or —C═N—NH(t-butyl) (or the like) and W^{x}, Z^{q1} and X¹ are as hereinbefore defined, with a compound of formula XXVIII,

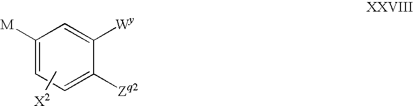

wherein M represents hydrogen or an appropriate alkali metal group (e.g. sodium, potassium or, especially, lithium), a —Mg-halide or a zinc-based group, or, a bromo group, and X², Z^{q2} and W^{y} are as hereinbefore defined, under reaction conditions known to those skilled in the art. For example in the case of reaction of a compound of formula XXVII in which T^{x} represents —C(O)Cl with a compound of formula XXVIII in which M represents hydrogen, in the presence of an appropriate Lewis acid. In the case where M represents an appropriate alkali metal group, a —Mg-halide or a zinc-based group, under reaction conditions such as those hereinbefore described in respect of preparation of compounds of formula II or VI (process step (IV) above) and preparation of compounds of formula XIII. In the case of a reaction of a compound of formula XXVII in which T^{x} represents —C═N—NH(t-butyl) (or the like) with a compound of formula XXVIII in which M represents bromo, under reaction conditions such as those described in Takemiya et al, J. Am. Chem. Soc. 128, 14800 (2006).

For compounds of formula XXV in which T represents —CH(OH)—, reaction of a compound corresponding to a compound of formula XXVII, but in which T^{x} represents —C(O)H, with a compound of formula XXVIII as defined above, under reaction conditions such as those hereinbefore described in respect of preparation of compounds of formula XXV in which T represents —C(O)—.

Compounds of formula XXIV may be prepared by reaction of a compound of formula XXIX,

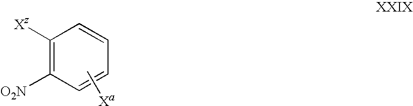

wherein X^{z} represents fluoro or bromo and X^{a} is as hereinbefore defined, under standard conditions, for example when X^{z} represents fluoro, in the presence of an appropriate source of cyanide ions (e.g. KCN) under standard nucleophilic aromatic substitution reaction conditions or, when X^{z} represents bromo, under palladium catalysed cyanation reaction conditions.

Compounds of formula XXVIB may be prepared by reaction of a compound of formula XXX,

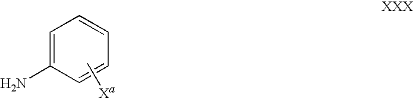

wherein X^{a} is as hereinbefore defined, with chloral hydrate, hydroxylamine hydrochloride, sodium sulfate and hydrochloric acid, followed by reaction in the presence of concentrated sulfuric acid, for example as described in the Sheibley et al journal article referenced herein.

Compounds of formulae III, IV, V, VII, VIII, IX, X, XI, XII, XIII, XIV, XVIII, XXI, XXVI, XXVIA, XXVII, XXVIII, XXIX and XXX (as well as other compounds, e.g. some compounds of formulae XV, XXIII and XXIV) are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991.

The substituents X¹, X², R^{x}, R^{y}, R^{w}, R^{z} Y¹ and Y² in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. Such reactions may result in the formation of a symmetric or asymmetric final compound of the invention or intermediate. The precursor groups can be changed to a different such group, or to the groups defined in formula I, at any time during the reaction sequence. For example, in cases where R^{x} and/or R^{y} does not initially represent hydrogen (so providing at least one ester functional group), the skilled person will appreciate that at any stage during the synthesis (e.g. the final step), the relevant R^{x} and/or R^{y}-containing group may be hydrolysed to form a carboxylic acid functional group (i.e. a group in which R^{x} and/or R^{y} represent hydrogen). In this respect, the skilled person may also refer to “Comprehensive Organic Functional Group Transformations” by A. R. Katritzky, O. Meth-Cohn and C. W. Rees, Pergamon Press, 1995.

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined but without proviso (c), for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which any of R^{x} and R^{y} are other than hydrogen, so forming an ester group) may possess no or minimal pharmacological activity as such, but may be administered parenterally or orally, and thereafter be metabolised in the body to form compounds of the invention that possess pharmacological activity as such (including, but not limited to, corresponding compounds of formula I, in which R^{x} and/or R^{y} represent hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the test described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III, thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterized by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Where a condition has an inflammatory component associated with it, or a condition characterized by inflammation as a symptom, the skilled person will appreciate that compounds of the invention may be useful in the treatment of the inflammatory symptoms and/or the inflammation associated with the condition.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulo-nephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined but without provisos (a) to (c), to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as hereinbefore defined but without proviso (c), in admixture with a pharmaceutically acceptable adjuvant, diluent or carrier.

Preferred pharmaceutical formulations include those in which the active ingredient is present in at least 1% (such as at least 10%, preferably in at least 30% and most preferably in at least 50%) by weight. That is, the ratio of active ingredient to the other components (i.e. the addition of adjuvant, diluent and carrier) of the pharmaceutical composition is at least 1:99 (e.g. at least 10:90, preferably at least 30:70 and most preferably at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. thromboxane receptor (TP) antagonists or, preferably, leukotriene receptor antagonists (LTRAs), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activating protein), immunosuppressants and sulphasalazine and related compounds and/or other therapeutic agents that are useful in the treatment of inflammation).

According to a further aspect of the invention, there is provided a combination product comprising:
(A) a compound of the invention, as hereinbefore defined but without provisos (a) to (c); and
(B) another therapeutic agent that is useful in the treatment of a respiratory disorder and/or inflammation,
   wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined but without provisos (a) to (c), another therapeutic agent that is useful in the treatment of a respiratory disorder and/or inflammation, and a pharmaceutically-acceptable adjuvant, diluent or carrier; and

(2) a kit of parts comprising components:
(a) a pharmaceutical formulation including a compound of the invention, as hereinbefore defined without provisos (a) to (c), in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or

(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise.

### Biological Test

In the assay LTC₄ synthase catalyses the reaction where the substrate LTA₄ methyl ester is converted to LTC₄ methyl ester. Recombinant human LTC₄ synthase is expressed in Piccia pastoralis and the purified enzyme is dissolved in 25 mM Tris-buffer pH 7.8 and stored at −20° C. The assay is performed in phosphate buffered saline (PBS) pH 7.4, supplemented with 5 mM glutathione (GSH). The reaction is terminated by addition of acetonitrile/MeOH/acetic acid (50/50/1). The assay is performed at it in 96-well plates. Analysis of the formed LTC₄ methyl ester is performed with reversed phase HPLC (Waters 2795 utilizing an Onyx Monolithic C18 column). The mobile phase consists of acetonitrile/MeOH/H₂O (32.5/30/37.5) with 1% acetic acid pH adjusted with NH₃ to pH 5.6, and absorbance measured at 280 nm with a Waters 2487 UV-detector.

The following is added chronologically to each well:
1. 50 μl assay buffer, PBS with 5 mM GSH.
2. 0.5 μl inhibitor in DMSO.
3. 2 μl LTC₄ synthase in PBS. The total protein concentration in this solution is 0.025 mg/ml. Incubation of the plate at room temperature for 10 minutes.
4. 0.5 μl LTA₄ methyl ester. Incubation of the plate at it for 1 min.
5. 50 μl stop solution.
   80 μl of the incubation mixture is analysed with HPLC.

### EXAMPLES

The invention is illustrated by way of the following examples, in which the following abbreviations may be employed:

BINAP 2,2′-bis(diphenylphosphino)-1,1′-binaphthyl
DMF dimethylformamide
EtOAc ethyl acetate
MeOH methanol
NMR nuclear magnetic resonance
rt room temperature
rx reflux
THF tetrahydrofuran

Chemicals specified in the synthesis of the compounds in the examples were commercially available from, e.g. Sigma-Aldrich Fine Chemicals, Maybridge or Acros Organics.

### Starting Materials I-V

### 5,5′-Methylenebis(salicylic acid) (4,4′-dihydroxydiphenylmethane-3,3′-dicarboxylic acid; CAS number: 122-25-8) (I)

The starting material 5,5′-methylenebis(salicylic acid) (I) is commercially available (from e.g. Acros Organics).

### Preparation of dimethyl 5,5′-methylenebis(2-hydroxybenzoate) (II)

Concentrated H₂SO₄ (2 mL) was added dropwise to a stirred suspension of I (7.2 g) in MeOH (20 mL) at rt. After stirring at rx for 10 h, the mixture was allowed to cool to rt before EtOAc (20 mL) was added. The mixture was washed with NaHCO₃ (aq, sat) until the washings turned weakly basic. The organic phase was dried (Na₂SO₄), concentrated and purified by chromatography, providing the title compound (II) in 1.3 g (16.4%) yield.

### Preparation of dimethyl 5,5′-methylenebis(2-(trifluoromethylsulfonyloxy)-benzoate) (III)

A suspension of II (0.79 g, 2.50 mmol) in toluene (10 mL) was mixed with K₃PO₄ (aq, 3 g in 10 mL water), cooled to 0° C., before triflic anhydride (1.7 g, 6.0 mmol) was added dropwise whilst stirring vigorously and maintaining the reaction temperature below 10° C. The mixture was stirred 2 h at rt, the layers were separated and the organic phase washed with water and concentrated. The residue was purified by chromatography, affording the title compound (III) in 0.786 g (54%) yield.

### Preparation of dimethyl 5,5′-methylenebis(2-aminobenzoate) (IV)

To a stirred solution of 60.5 g methyl 2-aminobenzoate, HCl (aq, sat, 144 mL) and water (500 mL) was slowly added formaldehyde (aq, 3%, 155 mL) at 50° C. over a period of 20 mins. The solution was heated with stirring at 80° C. for 6 h. After cooling to room temperature, the solution was neutralised with ammonia and a precipitation was formed which was filtered off, washed with water, dried and recrystallized from EtOH to give the product (IV) as a yellowish powder in 42.1 g (86%).

### Preparation of 5,5′-methylenebis(2-amino-3-chlorobenzoic acid) (V)

To a stirred mixture of 2-amino-3-chlorobenzoic acid (1.06 g, 6.20 mmol), water (20 mL), and HCl (aq, 37%, 4 mL) at 50° C. was added 3% aqueous formaldehyde (93.10 mg, 3.10 mmol). The mixture was heated at 70° C. and stirred for 4 h. After cooling, the white precipitate was filtered off, washed with water and MeOH to afford the title compound (952 mg, 86%) as a white solid. ¹H NMR (DMSO-d₆, 400 MHz), δ 7.59-7.55 (2H, m), 7.38-7.34 (2H, m), 3:69 (2H, s). ¹³C δ 168.6, 144.9, 133.8, 129.9, 127.9, 118.8, 111.4, 37.8.

5,5′-methylenebis(2-aminobenzoic acid) (VI) (which is commercially available from e.g. Maybridge or, alternatively, may be prepared as described in the literature, e.g. Bioorg. Med. Chem., 2006, 14, 2209) may be esterified under standard conditions known to those skilled in the art (which may involve the use of protecting groups) in order to give dimethyl 5,5′-methylenebis(2-aminobenzoate) (IV).

### General Methods Producing Exemplified Compounds 1-47

General method A for diarylamine coupling of III to produce dimethyl 5,5′-methylenebis(2-(arylamino)benzoate) VII, followed by hydrolysis to produce 5,5′-methylenebis(2-(arylamino)benzoic acid) VIII:

A mixture of compound III (0.196 g, 1 equiv), arylamine (1.2 equiv), Cs₂CO₃ (308 mg, 1.4 equiv), Pd(OAc)₂ (7.6 mg, 0.05 equiv), and BINAP (32 mg, 0.075 equiv) in toluene (1.34 mL) was stirred at 100° C. for 10 h. The mixture was filtered, concentrated and the residue purified by chromatography to furnish compound VII (see the yield of the ‘ester’ in Table 1). A mixture of VII (1 equiv), NaOH (aq, 2M, 2.8 mL) and dioxane (5.6 mL) was stirred for 5 h at 120° C., cooled to rt, acidified with HCl (aq, 10%, pH˜2-5), and extracted with EtOAc. The combined extracts were dried (Na₂SO₄) and concentrated to furnish compound VIII (see the yield of the acid in Table 1).

General method B for diarylamine coupling of IV to produce dimethyl 5,5′-methylenebis(2-(arylamino)benzoate) VII, followed by hydrolysis to produce 5,5′-methylenebis(2-(arylamino)benzoic acid) VIII:

An oven-dried vessel was charged with compound IV (0.157 g, 0.5 mmol), arylboronic acid (2 mmol), Cu(OAc)₂ (184 mg, 1 mmol), pyridine (82 μL, 1 mmol), triethylamine (140 μL, 1 mmol), dichloromethane (15 mL) and 4 Å molecular sieves. The mixture was stirred at rt for 4 days, filtered through Celite® and purified by chromatography, furnishing di- and mono-arylated compounds. Subsequent hydrolysis was carried out in accordance with the procedure described in Method A.

General method C for diarylamine coupling of IV to produce dimethyl 5,5′-methylenebis(2-(arylamino)benzoate) VII, followed by hydrolysis to produce 5,5′-methylenebis(2-(arylamino)benzoic acid) VIII:

A mixture of compound IV (0.314 g, 1 mmol), copper(I)tris(triphenyl-phosphine)bromide (372 mg, 0.4 mmol), Cs₂CO₃ (977 mg, 3 mmol), and toluene (15 mL) was heated under argon at 110° C. for 5 min. The aryl iodide (2 mmol) was added via syringe and the mixture was stirred at 110° C. for 1-2 days. After cooling, the mixture was filtered through a small pad of silica gel, concentrated and purified by chromatography, furnishing the di-arylated compound as the minor product and the mono-arylated as the major product. The subsequent hydrolysis was carried out in accordance with the procedure described in Method A.

General method D for diarylamine coupling of IV to produce dimethyl 5,5′-methylenebis(2-(arylamino)benzoate) VII, followed by hydrolysis to produce 5,5′-methylenebis(2-(arylamino)benzoic acid) VIII.

An oven-dried vessel was charged with compound IV (0.314 g, 1.0 mmol), Pd₂(dba)₃ (23 mg, 0.025 mmol), Cs₂CO₃ (0.762 g, 2.34 mmol), (rac)-BINAP (23.4 mg, 0.037 mmol), aryl bromide (1.67 mmol) and toluene (3 mL) under argon atmosphere. The mixture was heated at 100° C. for 2-4 days. If needed, a second addition of the reactants, except compound IV, was made in order to increase the conversion. The mixture was cooled, filtered, and purified by chromatography, furnishing di- and mono-arylated compounds. Subsequent hydrolysis was carried out in accordance with the procedure described in Method A.

General method E for di-alkylation of VII to produce dimethyl 5,5′-methylenebis(2-(aryl(alkyl)amino)benzoate) IX, followed by hydrolysis to produce 5,5′-methylenebis(2-(aryl(alkyl)amino)benzoic acid) X.

Compound VII (0.18 mmol) was added to a mixture of NaH (24 mg, 0.8 mmol, 80% in mineral oil) and DMF (2 mL). The mixture was stirred at it for 20 min. Alkyl halide (0.8 mmol) was added and the mixture was stirred at rt for 24 h. The mixture was diluted with water (12 mL) and acidified to ˜pH 6 with HCl (aq, 1M). Extractive work-up (water, EtOAc) and purification by chromatography gave the desired compounds IX. Subsequent hydrolysis was carried out in accordance with the procedure described in Method A. Purification by chromatography furnished compounds X.

General method F for mono-alkylation of VII to produce 2-[alkyl-(aryl)-amino]-5-[4-(arylamino)-3-methoxycarbonyl-benzyl]-benzoic acid methyl ester XI, followed by hydrolysis to produce 2-[alkyl-(aryl)-amino]-5-[3-carboxy-4-(arylamino)-benzyl]-benzoic acid XII.

To a stirred solution of VII (0.63 g, 1.2 mmol) in DMF (60 ml), n-butyl iodide (0.40 mL, 3.5 mmol) and NaH (47 mg, 1.2 mmol, 60% suspension in mineral oil) were added and the reaction mixture was stirred at room temperature for 1 h. The addition of water followed by an extractive workup (EtOAc, water, brine) and purification by chromatography gave the desired compounds XI (see the yield of the ester given in Table 1). A mixture of XI (0.58 g, 0.97 mmol), NaOH (0.28 g, 6.9 mmol), MeOH (25 mL) and water (2 mL) was stirred for 3 h at reflux, cooled to rt, concentrated in vacuo, diluted with brine (20 mL) acidified with HCl (1M, pH ˜2-5), and extracted with EtOAc. The combined extracts were dried (Na₂SO₄), concentrated and purified by column chromatography to furnish compound XII (see the yield of the acid in Table 1).

TABLE 1
Compounds of Examples 1-42 using general Method A-F

| | | | | Yield (%) | |
|---|---|---|---|---|---|
| No. | Chemical name | Method | Substrate | ester | acid |
| 1 | dimethyl 5,5′-methylenebis(2-(3-chloro- | A | 3-chloro-2- | 55 | — |
| | 2-methylphenylamino)benzoate) | | methylaniline | | |
| 2 | 5,5′-methylenebis(2-(3-chloro-2- | A | 3-chloro-2- | 55 | 93 |
| | methylphenylamino)benzoic acid) | | methylaniline | | |
| 3 | 5,5′-methylenebis(2-(3,5-difluoro- | B | 3,5-difluoro | 65 | 75 |
| | phenylamino)benzoic acid) | | phenylboronic | | |
| | | | acid | | |
| 4 | 2-amino-5-(3-carboxy-4-(3,5-difluoro- | B | 3,5-difluoro- | 33 | 65 |
| | phenylamino)benzyl)benzoic acid | | phenylboronic | | |
| | | | acid | | |
| 5 | 5,5′-methylenebis(2-(3-chloro-4- | B | 3-chloro-4- | 28 | 75 |
| | fluorophenylamino)benzoic acid) | | fluorophenyl- | | |
| | | | boronic acid | | |
| 6 | 2-amino-5-(3-carboxy-4-(3-chloro-4- | C | 3-chloro-4- | 27 | 53 |
| | fluorophenylamino)benzyl)benzoic acid | | fluoro-1-iodo- | | |
| | | | benzene | | |
| 7 | 5,5′-methylenebis(2-(4-bromo-2- | C | 4-bromo-2- | 11 | 65 |
| | fluorophenylamino)benzoic acid) | | fluoro-1- | | |
| | | | iodobenzene | | |
| 8 | 2-amino-5-(4-(4-bromo-2-fluorophenyl- | C | 4-bromo-2- | 41 | 41 |
| | amino)-3-carboxybenzyl)benzoic acid | | fluoro-1-iodo- | | |
| | | | benzene | | |
| 9 | 5,5′-methylenebis(2-(2-carboxyphenyl- | C | methyl 2-iodo- | 8 | 58 |
| | amino)benzoic acid) | | benzoate | | |
| 10 | 2-amino-5-(3-carboxy-4-(2-carboxy- | C | methyl 2-iodo- | 26 | 55 |
| | phenylamino)benzyl)benzoic acid | | benzoate | | |
| 11 | 5,5′-methylenebis(2-(4-fluorophenyl- | B | 4-fluorophenyl | 15 | 76 |
| | amino)benzoic acid) | | boronic acid | | |
| 12 | 2-amino-5-(3-carboxy-4-(4-fluoro- | B | 4-fluorophenyl | 45 | 68 |
| | phenylamino)benzyl)benzoic acid | | boronic acid | | |
| 13 | 5,5′-methylenebis(2-(3,4-difluoro- | B | 3,4-difluoro- | 39 | 83 |
| | phenylamino)benzoic acid) | | phenylboronic | | |
| | | | acid | | |
| 14 | 2-amino-5-(3-carboxy-4-(3,4-difluoro- | B | 3,4-difluoro- | 43 | 58 |
| | phenylamino)benzyl)benzoic acid | | phenylboronic | | |
| | | | acid | | |
| 15 | 5,5′-methylenebis(2-(3-methoxyphenyl- | C | 1-iodo-3- | 24 | 57 |
| | amino)benzoic acid) | | methoxy | | |
| | | | benzene | | |
| 16 | 2-amino-5-(3-carboxy-4-(3-methoxy- | C | 1-iodo-3- | 42 | 80 |
| | phenylamino)benzyl)benzoic acid | | methoxy | | |
| | | | benzene | | |
| 17 | 2-amino-5-(3-carboxy-4-(2-fluoro- | B | 2-fluorophenyl | 10 | 27 |
| | phenylamino)benzyl)benzoic acid | | boronic acid | | |
| 18 | 5,5′-methylenebis(2-(3-fluoruphenyl- | B | 3-fluorophenyl | 12 | 67 |
| | amino)benzoic acid) | | boronic acid | | |
| 19 | 2-amino-5-(3-carboxy-4-(3- | B | 3-fluorophenyl | 29 | 82 |
| | fluorophenylamino)benzyl)benzoic acid | | boronic acid | | |
| 20 | 5,5′-methylenebis(2-(3-chlorophenyl- | B | 3-chlorophenyl | 25 | 84 |
| | amino)benzoic acid) | | boronic acid | | |
| 21 | 2-amino-5-(3-carboxy-4-(3- | B | 3-chlorophenyl | 41 | 65 |
| | chlorophenylamino)benzyl)benzoic acid | | boronic acid | | |
| 22 | 2-amino-5-(3-carboxy-4-(o-tolyl-amino)- | B | o-tolylboronic | 11 | 62 |
| | benzyl)benzoic acid | | acid | | |
| 23 | 2-amino-5-(3-carboxy-4-(3-chloro-2- | C | 1-chloro-3- | 35 | 68 |
| | methylphenylamino)benzyl)benzoic | | iodo-2-methyl- | | |
| | acid | | benzene | | |
| 24 | 2-amino-5-(3-carboxy-4-(4-methoxy- | C | 1-iodo-4- | 46 | 59 |
| | phenylamino)benzyl)benzoic acid | | methoxy | | |
| | | | benzene | | |
| 25 | 2-amino-5-(3-carboxy-4-(2-methoxy- | C | 1-iodo-2- | 24 | 62 |
| | phenylamino)benzyl)benzoic acid | | methoxy | | |
| | | | benzene | | |
| 26 | 5,5′-methylenebis(2-(2-methoxyphenyl- | C | 1-iodo-2- | 10 | 39 |
| | amino)benzoic acid) | | methoxy | | |
| | | | benzene | | |
| 27 | 2-amino-5-(3-carboxy-4-(4-isopropoxy- | B | 4-isopropoxy- | 70 | 33 |
| | phenylamino)benzyl)benzoic acid | | phenylboronic | | |
| | | | acid | | |
| 28 | 2-amino-5-(3-carboxy-4-(naphthalen-2- | B | naphthalen-2- | 31 | 39 |
| | ylamino)benzyl)benzoic acid | | ylboronic acid | | |
| 29 | 5,5′-methylenebis(2-(naphthalen-2-yl- | B | naphthalen-2- | 9 | 80 |
| | amino)benzoic acid) | | ylboronic acid | | |
| 30 | 5,5′-methylenebis(2-(phenylamino)- | B | phenylboronic | 32 | 78 |
| | benzoic acid) | | acid | | |
| 31 | 2-amino-5-(3-carboxy-4-(naphthalen-1- | B | naphthalen-1- | 22 | 41 |
| | ylamino)benzyl)benzoic acid | | ylboronic acid | | |
| 32 | 5,5′-methylenebis(2-(4-(trifluoro- | D | 1-bromo-4-(tri- | 61 | 78 |
| | methyl)phenylamino)benzoic acid) | | fluoromethyl)- | | |
| | | | benzene | | |
| 33 | 5,5′-methylenebis(2-(4-t-butyl- | D | 1-bromo-4-t- | 16 | 31 |
| | phenylamino)benzoic acid) | | butylbenzene | | |
| 34 | 5,5′-methylenebis(2-(3,4-dichloro- | B | 3,4-dichloro- | 47 | 70 |
| | phenylamino)benzoic acid) | | phenylboronic | | |
| | | | acid | | |
| 35 | 5,5′-methylenebis(2-(2,4-difluoro- | D | 1-bromo-2,4- | 75 | 86 |
| | phenylamino)benzoic acid) | | difluoro- | | |
| | | | benzene | | |
| 36 | 5,5′-methylenebis(2-((3,4-difluoro- | E | dimethyl 5,5′- | 64 | 60 |
| | phenyl)methylamino)benzoic acid) | | methylenebis- | | |
| | | | (2-(3,4-difluoro | | |
| | | | phenylamino) | | |
| | | | benzoate) | | |
| 37 | 5,5′-methylenebis(2-((4-bromo-2- | E | dimethyl 5,5′- | 63 | 80 |
| | fluorophenyl)methylamino)benzoic | | methylenebis- | | |
| | acid) | | (2-(4-bromo-2- | | |
| | | | fluorophenyl | | |
| | | | amino) | | |
| | | | benzoate) | | |
| 38 | 5,5′-methylenebis(2-(4- | D | 1-bromo-4- | 25 | 48 |
| | chlorophenylamino)benzoic acid) | | chlorobenzene | | |
| 39 | 5,5′-methylenebis(2-(p-tolylamino)- | D | 1-bromo-4- | 20 | 80 |
| | benzoic acid) | | methylbenzene | | |
| 40 | 5,5′-methylenebis(2-(3-fluoro-4-(tri- | D* | 4-bromo-2- | 63 | 75 |
| | fluoromethoxy)phenylamino)benzoic | | fluoro-1- | | |
| | acid) | | (trifluoromethoxy) | | |
| | | | benzene | | |
| 41 | 2-[butyl-(3,4-difluorophenyl)-amino]-5- | F | 5,5′- | 86 | 12 |
| | [3-carboxy-4-(3,4-difluorophenylamino)- | | methylenebis(2- | | |
| | benzyl]-benzoic acid | | (3,4-difluoro- | | |
| | | | phenylamino)benzoic | | |
| | | | acid) | | |
| 42 | 5-{3-carboxy-4-[(3,4-difluorophenyl)-(4- | E | 5,5′- | 79 | 21^{#} |
| | methylbenzyl)amino]-benzyl}-2-[(3,4- | | methylenebis(2- | | |
| | difluorophenyl)-(4-methylbenzyl)- | | (3,4-difluoro- | | |
| | amino]-benzoic acid | | phenylamino)benzoic | | |
| | | | acid) | | |
| | | | methyl ester | | |

*Pd(OAc)₂ was used as the palladium source (5 mol-%)
^{#}Hydrolyzed according to the procedure in General Method F.

TABLE 2
Spectroscopic data of the compounds of Examples 1-42

| Example | |
|---|---|
| No. | ¹H NMR (DMSO-d₆, 200 MHz), δ: |
| 1 | 2.32 (s, 6H); 3.79 (s, 2H); 3.89 (s, 6H); 6.80 (d, J = 8.6 Hz, 2H); |
| | 7.02-7.24 (m, 8H); 7.79 (d, J = 2.1 Hz, 2H); 9.21 (br s, 2H). |
| 2 | 2.24 (s, 6H); 3.79 (s, 2H); 6.83 (d, J = 8.6 Hz, 2H); 7.17-7.32 (m, 8H); |
| | 7.76 (d, J = 2.1 Hz, 2H); 9.45 (br s, 2H); 12.9-13.3 (br s, 2H). |
| 3 | 3.89 (s, 2H); 6.64-6.79 (m, 2H); 6.79-6.97 (m, 4H); 7.30-7.44 (m, 4H); |
| | 7.80 (s, 2H); 9.51 (s, 2H); 13.0-13.5 (br s, 2H). |
| 4 | 3.75 (s, 2H); 6.61-6.78 (m, 2H); 6.78-6.94 (m, 2H); 7.11 (dd, J = 8.5 and |
| | 2.2 Hz, 1H); 7.27-7.40 (m, 2H,); 7.54 (d, J = 2.2 Hz, 1H); 7.73 (d, J = 1.8 Hz, |
| | 1H); 8.1-8.9 (br s, 2H); 9.47 (s, 1H); 12.5-13.5 (br s, 2H). |
| 5 | 3.82 (s, 2H); 7.09-7.37 (m, 8H); 7.42 (dd, J = 6.8 and 2.5 Hz, 2H); |
| | 7.76 (unresolved d, 2H); 9.3-9.6 (br s, 2H); 12.7-13.4 (br s, 2H). |
| 6 | 3.71 (s, 2H); 6.67 (d, J = 7.9 Hz, 1H); 7.09 (dd, J = 8.3 and 2.2 Hz, 1H); |
| | 7.12-7.40 (m, 4H); 7.41 (dd, J = 6.5 and 2.5 Hz, 1H); 7.52 (d, J = 2.2 Hz, |
| | 1H); 7.70 (d, J = 2.2 Hz, 1H); 8.1-8.9 (br, 2H); 9.2-9.6 (br s, 1H); |
| | 12.5-13.5 (br s, 2H). |
| 7 | 3.85 (s, 2H); 7.10 (d, J = 8.8 Hz, 2H); 7.25-7.38 (m, 4H); 7.45 (dd, J = 8.5 |
| | and 8.8 Hz, 2H); 7.60 (dd, J = 10.7 and 2.1 Hz, 2H); 7.78 (d, J = 2.1 Hz, |
| | 2H); 9.55 (s, 2H); 12.8-13.8 (br s, 2H). |
| 8 | 3.72 (s, 2H); 6.65 (d, J = 8.4 Hz, 1H); 7.06 (dd, J = 8.4 and 1.8 Hz, 1H); |
| | 7.07 (d, J = 8.4 Hz, 1H); 7.25 (dd, J = 8.4 and 1.8 Hz, 1H); 7.30 (d, J = 8.4 Hz, |
| | 1H); 7.41 (dd, J = 9.0 and 8.4 Hz, 1H); 7.50 (d, J = 1.8 Hz, 1H); |
| | 7.57 (dd, J = 10.8 and 1.8 Hz, 1H); 7.70 (d, J = 1.8 Hz, 1H); 8.1-8.8 (br s, 2H); |
| | 9.50 (s, 1H); 12.6-13.5 (br s, 2H). |
| 9 | 3.90 (s, 2H); 6.80-7.02 (m, 2H); 7.23-7.61 (m, 8H); 7.79 (s, 2H); |
| | 7.89 (d, J = 7.7 Hz, 2H); 10.3-11.1 (br s, 2H); 12.4-13.4 (br s, 4H). |
| 10 | 3.76 (s, 2H); 6.69 (d, J = 8.4 Hz, 1H); 6.83-6.97 (m, 1H); 7.11 (dd, J = 8.4 |
| | and 1.8 Hz, 1H); 7.27 (dd, J = 8.4 and 1.8 Hz, 1H); 7.34-7.49 (m, 3H); |
| | 7.56 (d, J = 1.8 Hz, 1H); 7.71 (d, J = 1.8 Hz, 1H); 7.89 (d, J = 8.0 Hz, 1H); |
| | 7.5-9.8 (br s, 3H); 10.2-11.2 (br s, 1H); 11.3-14.3 (br s, 2H). |
| 11 | 3.78 (s, 2H); 7.04 (d, J = 8.8 Hz, 2H); 7.09-7.32 (m, 10H); 7.73 (d, J = 1.9 Hz, |
| | 2H); 9.41 (s, 2H); 12.9-13.1 (br s, 2H). |
| 12 | 3.69 (s, 2H); 6.62-6.72 (m, 1H); 6.98-7.30 (m, 7H); 7.51 (d, J = 2.1 Hz, |
| | 1H); 7.69 (d, J = 2.1 Hz, 1H); 8.1-8.9 (br s, 2H); 9.42 (s, 1H); |
| | 12.2-13.4 (br s, 2H). |
| 13 | 3.82 (s, 2H); 6.98-7.10 (m, 2H); 7.18 (d, J = 8.5 Hz, 2H); 7.23-7.45 (m, |
| | 6H); 7.76 (d, J = 1.9 Hz, 2H); 9.44 (s, 2H); 13.0-13.3 (br s, 2H). |
| 14 | 3.72 (s, 2H); 6.67 (d, J = 8.5 Hz, 1H); 6.96-7.05 (m, 1H); 7.09 (dd, J = 8.5 |
| | and 2.1 Hz, 1H); 7.16 (d, J = 8.5 Hz, 1H); 7.21-7.43 (m, 3H); 7.52 (d, |
| | J = 2.1 Hz, 1H); 7.71 (d, J = 1.8 Hz, 1H); 8.2-8.7 (br s, 2H); 9.42 (s, 1H); |
| | 12.8-13.3 (br s, 2H). |
| 15 | 3.73 (s, 6H); 3.81 (s, 2H); 6.55-6.64 (m, 2H); 6.73-6.82 (m, 4H); |
| | 7.20 (d, J = 8.1 Hz, 2H); 7.24-7.30 (m, 4H); 7.73-7.78 (m, 2H); 9.3-9.7 (br s, |
| | 2H); 12.5-13.6 (br s, 2H). |
| 16 | 3.71 (s, 2H); 3.73 (s, 3H); 6.55-6.63 (m, 1H); 6.68 (d, J = 8.5 Hz, 1H); |
| | 6.72-6.81 (m, 2H); 7.09 (dd, J = 8.5 and 2.1 Hz, 1H); 7.19 (d, J = 8.0 Hz, |
| | 1H): 7.22-7.27 (m, 2H); 7.53 (d, J = 2.1 Hz, 1H); 7.70 (s, 1H); 7.9-9.1 (br |
| | s, 2H); 9.47 (br s, 1H); 11.9-13.8 (br s, 2H). |
| 17 | 3.72 (s, 2H); 6.67 (d, J = 8.8 Hz, 1H); 7.00-7.35 (m, 6H); 7.46 (dd, J = 8.6 |
| | and 1.8 Hz, 1H); 7.53 (d, J = 1.8 Hz, 1H); 7.72 (d, J = 1.8 Hz, 1H); |
| | 8.2-8.8 (br s, 2H); 9.53 (s, 1H); 13.0-13.3 (br s, 2H). |
| 18 | 3.85 (s, 2H); 6.72-6.85 (m, 2H); 6.97-7.11 (m, 4H); 7.24-7.40 (m, 6H); |
| | 7.78 (s, 2H); 9.55 (s, 2H); 12.6-13.5 (br s, 2H). |
| 19 | 3.73 (s, 2H); 6.68 (d, J = 8.4 Hz, 1H); 6.72-6.84 (m, 1H); 6.97-7.14 (m, |
| | 3H); 7.22-7.38 (m, 3H); 7.53 (d, J = 2.2 Hz, 1H); 7.72 (unresolved d, 1H); |
| | 7.9-8.9 (br s, 2H); 9.50 (s, 1H); 12.0-13.5 (br s, 2H). |
| 20 | 3.85 (s, 2H); 6.97-7.07 (m, 2H); 7.11-7.21 (m, 2H); 7.21-7.38 (m, 8H); |
| | 7.78 (d, J = 1.8 Hz, 2H); 9.49 (s, 2H); 12.9-13.4 (br s, 2H). |
| 21 | 3.73 (s, 2H); 6.68 (d, J = 8.5 Hz, 1H); 6.96-7.05 (m, 1H); 7.10 (dd, J = 8.5 |
| | and 2.1 Hz, 1H); 7.10-7.19 (m, 1H); 7.20-7.36 (m, 4H); 7.53 (d, J = 2.1 Hz, |
| | 1H); 7.72 (unresolved d, 1H); 8.1-8.8 (br s, 2H); 9.47 (s, 1H); |
| | 12.0-13.7 (br s, 2H). |
| 22 | 2.19 (s, 3H); 3.69 (s, 2H); 6.67 (d, J = 8.4 Hz, 1H); 6.88 (d, J = 8.4 Hz, |
| | 1H); 6.96-7.33 (m, 6H); 7.51 (d, J = 2.1 Hz, 1H); 7.69 (d, J = 2.1 Hz, 1H); |
| | 8.1-8.8 (br s, 2H); 9.39 (s, 1H); 12.7-13.3 (br s, 2H). |
| 23 | 2.26 (s, 3H); 3.70 (s, 2H); 6.67 (d, J = 8.5 Hz, 1H); 6.82 (d, J = 8.5 Hz, |
| | 1H); 7.08 (dd, J = 8.5 and 2.1 Hz, 1H); 7.15-7.32 (m, 4H); 7.52 (d, J = 2.1 Hz, |
| | 1H); 7.71 (d, J = 2.1 Hz, 1H); 7.9-8.9 (br s, 2H); 9.46 (s, 1H); |
| | 12.0-13.5 (br s, 2H). |
| 24 | 3.67 (s, 2H); 3.74 (s, 3H); 6.67 (d, J = 7.9 Hz, 1H); 6.84-6.99 (m, 3H); |
| | 7.03-7.24 (m, 4H); 7.51 (unresolved d, 1H); 7.66 (unresolved d, 1H); |
| | 7.9-8.9 (br s, 2H); 9.30 (br s, 1H); 11.5-13.7 (br s, 2H). |
| 25 | 3.70 (s, 2H); 3.81 (s, 3H); 6.67 (d, J = 8.5 Hz, 1H); 6.84-7.15 (m, 4H); |
| | 7.16-7.24 (m, 2H); 7.34 (unresolved dd, J = 7.8 Hz, 1H); |
| | 7.53 (unresolved d, 1H); 7.70 (unresolved d, 1H); 8.0-8.8 (br s, 2H); 9.53 (br |
| | s, 1H); 12.0-14.0 (br s, 2H). |
| 26 | 3.79 (s, 2H); 3.81 (s, 6H); 6.83-7.09 (m, 5H); 7.12-7.28 (m, 4H); |
| | 7.36 (d, J = 7.5 Hz, 2H); 7.54-7.65 (m, 1H); 7.74 (unresolved d, 2H); |
| | 9.3-9.7 (br s, 2H); 12.0-14.0 (br s, 2H). |
| 27 | 1.26 (d, J = 6.0 Hz, 6H; 3.67 (s, 2H); 4.55 (heptet, J = 6.0 Hz, 1H); |
| | 6.67 (d, J = 8.6 Hz, 1H); 6.84-6.97 (m, 3H); 7.03-7.17 (m, 4H); 7.53 (d, J = 2.3 Hz, |
| | 1H); 7.69 (d, J = 2.3 Hz, 1H); 8.0-9.0 (br s, 2H); 9.0-9.5 (s, 1H); |
| | 12.4-13.3 (br s, 1H). |
| 28 | 3.73 (s, 2H); 6.69 (d, J = 8.4 Hz, 1H); 7.11 (dd, J = 8.4 and 2.0 Hz, 1H); |
| | 7.23-7.50 (m, 5H); 7.55 (d, J = 1.8 Hz, 1H); 7.64-7.90 (m, 5H); |
| | 8.2-8.8 (br s, 2H); 9.70 (s, 1H); 12.6-13.4 (br s, 2H). |
| 29 | 3.86 (s, 2H); 7.29-7.49 (m, 10H); 7.67-7.91 (m, 10H); 9.73 (s, 2H); |
| | 13.17 (s, 2H). |
| 30 | 3.80 (s, 2H); 7.03 (t, J = 7.4 Hz, 2H); 7.15-7.39 (m, 12H); |
| | 7.75 (unresolved d, 2H); 9.51 (s, 2H); 12.9-13.2 (br s, 2H). |
| 31 | 3.70 (s, 2H); 6.68 (d, J = 8.6 Hz, 1H); 6.93 (d, J = 8.6 Hz, 1H); 7.09 (dd, |
| | J = 8.6 and 2.0 Hz, 1H); 7.18 (dd, J = 8.6 and 2.0 Hz, 1H); 7.43-7.60 (m, |
| | 5H); 7.68-7.78 (m, 2H); 7.90-8.02 (m, 2H); 8.1-8.8 (br s, 2H); |
| | 9.8-10.1 (br s, 2H). |
| 32 | 9.6 (2H, s) 7.81 (2H, d, J = 1.5 Hz) 7.55-7.64 (4H, m) 7.37-7.46 (4H, m) |
| | 7.28-7.37 (4H, m) 3.90 (2H, s). |
| 33 | 13.0 (2H, br s) 9.46 (2H, s) 7.73 (2H, d, J = 1.8 Hz) 7.29-7.39 (4H, m) |
| | 7.24 (2H, dd, J = 8.8 and 1.8 Hz) 7.08-7.19 (6H, m) 3.77 (2H, s) |
| | 1.27 (18H, s). |
| 34 | 12.7-13.5 (2H, br s) 9.46 (2H, s) 7.78 (2H, d, J = 1.8 Hz) 7.49 (2H, d, |
| | J = 8.7 Hz) 7.44 (2H, d, J = 2.5 Hz) 7.35 (2H, dd, J = 8.7 and 1.8 Hz) |
| | 7.27 (2H, d, J = 8.7 Hz) 7.19 (2H, dd, J = 8.7 and 2.5 Hz) 3.86 (2H, s). |
| 35 | 10.5-9.5 (2H, br s) 7.75 (2H, d, J = 1.9 Hz) 7.54-7.13 (6H, m) |
| | 7.10-6.95 (2H, m) 6.86 (2H, d, J = 8.5 Hz) 3.76 (2H, s). |
| 36 | 12.6-13.1 (2H, br s) 7.72 (2H, d, J = 1.6 Hz) 7.55 (2H, dd, J = 8.0 and 1.6 Hz) |
| | 7.27 (2H, d, J = 8.0 Hz) 7.03-7.20 (2H, m) 6.39-6.54 (2H, m) |
| | 6.08-6.22 (2H, m) 4.09 (2H, s) 3.14 (6H, s). |
| 37 | 3.16 (s, 6H); 3.93 (s, 2H); 6.85 (t, J = 8.9 Hz, 2H); 7.11-7.41 (m, 8H); |
| | 7.48 (d, J = 2.1 Hz, 2H); 12.5-1 2.7 (br s, 2H). |
| 38 | 12.9-13.3 (2H, br s) 9.49 (2H, s) 7.76 (2H, d, J = 2.0 Hz) 7.16-7.38 (12H, |
| | m) 3.82 (2H, s). |
| 39 | 12.9-13.1 (2H, br s) 9.42 (2H, s) 7.72 (2H, d, J = 2.0 Hz) 7.05-7.27 (12H, |
| | m) 3.77 (2H, s) 2.27 (6H, s). |
| 40 | 9.5 (2H, br s) 7.77 (2H, s) 7.44-7.24 (8H, m) 7.06 (2H, d, J = 9.3 Hz) |
| | 3.86 (2H, s). |
| 41 | 9.75-9.30 (1H, bs) 7.88-7.79 (1H, m) 7.65 (1H, d, J = 2.0 Hz) |
| | 7.52-7.00 (8H, m) 6.44-6.30 (1H, m) 6.16-6.04 (1H, m) 3.96 (2H, s) 3.48 (2H, t, |
| | J = 7.5 Hz) 1.60-1.41 (2H, m) 1.36-1.21 (2H, m) 0.85 (3H, t, J = 7.2 Hz). |
| 42 | 7.95 (2H, d, J = 2.0 Hz) 7.36 (2H, dd, J = 8.0 2.0 Hz) 7.19 (2H, d, J = 8.0 Hz) |
| | 7.09-6.93 (10H, m) 6.79-6.65 (2H, m) 6.64-6.53 (2H, m) 4.63 (4H, |
| | s) 4.03 (2H, s) 2.26 (6H, s) |

### General Procedure for Preparation of Unsymmetrical Diaryl-Substituted Compounds

Mono-arylated ester compounds were synthesised in accordance with Method C. The second arylation step was performed in accordance with Method B to furnish the desired unsymmetrical diaryl-substituted compounds, which compounds were then subjected to hydrolysis.

TABLE 3
Unsymmetrical Diaryl-substituted Compounds of Examples 43-47 using
general Method C followed by general Method B

| | | | 2nd arylating | | |
|---|---|---|---|---|---|
| | | 1st arylating | or alkylating | Yield (%) | |
| No. | Chemical name | agent | agent | ester | Acid |
| 43 | 2-(4-bromo-2-fluorophenylamino)-5- | 4-bromo-2- | 4-(ethoxy- | 10 | 67 |
| | (3-carboxy-4-(4-carboxyphenyl- | fluoro-1- | carbonyl)- | | |
| | amino)benzyl)benzoic acid | iodobenzene | phenyl | | |
| | | | boronic acid | | |
| 44 | 2-(4-bromo-2-fluorophenylamino)-5- | 4-bromo-2- | 3,5-difluoro- | 63 | 81 |
| | (3-carboxy-4-(3,5-difluorophenyl- | fluoro-1- | phenyl- | | |
| | amino)benzyl)benzoic acid | iodobenzene | boronic acid | | |
| 45 | 2-(4-bromo-2-fluorophenylamino)-5- | 4-bromo-2- | 3-chloro-4- | 70 | 46 |
| | (3-carboxy-4-(3-chloro-4- | fluoro-1- | fluorophenyl | | |
| | fluorophenylamino)benzyl)benzoic | iodobenzene | boronic acid | | |
| | acid | | | | |
| 46 | 5-(3-carboxy-4-(3,5-difluorophenyl- | 2-chloro-1- | 3,5-difluoro- | 52 | 84 |
| | amino)benzyl)-2-(3-chloro-4-fluoro- | fluoro-4- | phenyl- | | |
| | phenylamino)benzoic acid | iodobenzene | boronic acid | | |
| 47^{#} | 5-[3-carboxy-4-(4-cyclohexyl- | 3,4-dichloro- | 4-cyclohexyl | 68 | 62 |
| | phenylamino)-benzyl]-2-(3,4- | phenyl | boronic acid | | |
| | dichloro-phenylamino)-benzoic acid | boronic acid | | | |

^{#}Mono-arylated ester compound was synthesized in accordance with Method B.

TABLE 4
Spectroscopic data of the compounds of Examples 43-47

| No | ¹H NMR (DMSO-d₆, 200 MHz), δ: |
|---|---|
| 43 | 3.87 (s, 2H); 7.11 (d, J = 8.5 Hz, 1H); 7.17-7.27 (m, 2H); |
| | 7.28-7.51 (m, 5H); 7.61 (dd, J = 10.5 and 2.1 Hz, 1H); 7.76-7.90 |
| | (m, 4H); 9.56 (s, 1H); 9.67 (s, 1H); 12.3-12.8 (br s, 1H); 13.0-13.5 |
| | (br s, 2H). |
| 44 | 3.87 (s, 2H); 6.64-6.79 (m, 1H); 6.79-6.96 (m, 2H); 7.11 (d, J = 8.4 |
| | Hz, 1H); 7.28-7.53 (m, 6H); 7.61 (dd, J = 10.7 and 1.8 Hz, 1H); |
| | 7.80 (d, J = 2.0 Hz, 1H); 9.49 (s, 1H); 9.55 (s, 1H); 13.1-13.4 |
| | (br s, 2H). |
| 45 | 3.83 (s, 2H); 7.05-7.52 (m, 9H); 7.60 (dd, J = 10.6 and 2.1 Hz, 1H); |
| | 7.76 (d, J = 2.3 Hz, 1H); 7.78 (d, J = 1.9 Hz, 1H); 9.40 (s, 1H); 9.54 |
| | (s, 1H); 13.0-13.4 (br s, 2H). |
| 46 | 3.86 (s, 2H); 6.63-6.79 (m, 1H); 6.79-6.95 (m, 2H); 7.10-7.47 (m, |
| | 7H); 7.75-7.81 (unresolved d, 2H); 9.41 (s, 1H); 9.49 (s, 1H); |
| | 13.0-13.4 (br s, 2H). |
| 47 | 13.4-12.5 (2H, br s), 9.45 (2H, br s), 7.79-7.70 (2H, m), 7.48 (1H, d, |
| | J = 8.8 Hz), 7.43 (1H, d, J = 2.5 Hz), 7.37-7.05 (9H, m), 3.81 |
| | (2H, s), 2.41 (1H, s, overlap with DMSO), 1.88-1.60 (5H, m), |
| | 1.52-1.09 (5H, m). |

### General Methods Producing Exemplified Compounds 48-66

### General Method G for the Preparation of Examples 48-58, 61-64

A mixture of 5,5′-methylenebis(2-aminobenzoic acid) (VI) (250 mg, 0.873 mmol), arylisocyanate (2.10 mmol), and THF (20 mL) was stirred at rt overnight. The precipitate was collected (precipitation with n-hexane if necessary) and recrystallised from THF/n-hexane to give the title product as a solid.

### General Method H for Monocarbamidation for the Preparation of Examples 59-60

A mixture of VI (250 mg, 0.873 mmol), arylisocyanate (0.291 mmol), and THF (20 mL) (or dioxane (40 mL) in Examples 11-12) was stirred at rt overnight. The product was precipitated with n-hexane and washed with HCl (aq) to give the title compound as a solid after recrystallisation from MeOH or dioxane/water.

### General Method I for The Preparation of Examples 65-66

A mixture of V (100 mg, 0.282 mmol), arylisocyanate (0.676 mmol), Et₃N (77 mg, 0.762 mmol) and THF (10 mL) was stirred at rt overnight. The precipitate was collected and sonicated with HCl (aq., 2M). The solid was filtered off, washed with HCl (aq., 2M), water and THF to afford the title product as a solid.

TABLE 5
Compounds of Examples 48-66

| No. | Chemical name | Carbamoylating agent | Method | Yield (%) |
|---|---|---|---|---|
| 48 | 5,5′-methylenebis- | 3-chlorophenyl isocyanate | G | 67 |
| | (2-(3-(3-chlorophenyl)- | | | |
| | ureido)benzoic acid) | | | |
| 49 | 5,5′-methylenebis(2-(3- | phenyl isocyanate | G | 70 |
| | phenylureido)benzoic acid) | | | |
| 50 | 5,5′-methylenebis- | 3-cyanophenyl | G | 75 |
| | (2-(3-(3-cyanophenyl)- | isocyanate | | |
| | ureido)benzoic acid) | | | |
| 51 | 5,5′-methylenebis- | 3-acetylphenyl | G | 80 |
| | (2-(3-(3-acetylphenyl)- | isocyanate | | |
| | ureido)benzoic acid) | | | |
| 52 | 5,5′-methylenebis(2-(3- | m-tolyl isocyanate | G | 72 |
| | m-tolylureido)benzoic acid) | | | |
| 53 | 5,5′-methylenebis- | 4-nitrophenyl isocyanate | G | 89 |
| | (2-(3-(4-nitrophenyl)- | | | |
| | ureido)benzoic acid) | | | |
| 54 | 5,5′-methylenebis(2-(3- | o-tolyl isocyanate | G | 50 |
| | o-tolylureido)benzoic acid) | | | |
| 55 | 5,5′-methylenebis- | 3-methoxyphenyl isocyanate | G | 66 |
| | (2-(3-(3-methoxyphenyl)- | | | |
| | ureido) benzoic acid) | | | |
| 56 | 5,5′-methylenebis- | 4-chlorophenyl | G | 60 |
| | (2-(3-(4-chlorophenyl)- | isocyanate | | |
| | ureido)benzoic acid) | | | |
| 57 | 5,5′-methylenebis- | 2-fluorophenyl | G | 45 |
| | (2-(3-(2-fluorophenyl)- | isocyanate | | |
| | ureido)benzoic acid) | | | |
| 58 | 5,5′-methylenebis- | 4-bromophenyl | G | 14 |
| | (2-(3-(4-bromophenyl)- | isocyanate | | |
| | ureido)benzoic acid) | | | |
| 59 | 2-amino-5-(4-(3-(4-bromo- | 4-bromophenyl | H | 12 |
| | phenyl)ureido)-3-carboxy- | isocyanate | | |
| | benzyl)benzoic acid | | | |
| 60 | 2-amino-5-(3-carboxy- | 4-nitrophenyl isocyanate | H | 73 |
| | 4-(3-(4-nitrophenyl)ureido)- | | | |
| | benzyl)benzoic acid | | | |
| 61 | 5,5′-methylenebis(2-(3-(2- | 2-trifluoromethylphenyl | G | 54 |
| | (trifluoromethyl)phenyl)- | isocyanate | | |
| | ureido)benzoic acid) | | | |
| 62 | 5,5′-methylenebis- | 2,6-dichlorophenyl | G | 35 |
| | (2-(3-(2,6-dichlorophenyl)- | isocyanate | | |
| | ureido)benzoic acid) | | | |
| 63 | 5,5′-methylenebis- | 4-butoxyphenyl | G | 30 |
| | (2-(3-(4-butoxyphenyl)- | isocyanate | | |
| | ureido)benzoic acid) | | | |
| 64 | 5,5′-methylenebis- | 2-phenoxyphenyl | G | 16 |
| | (2-(3-(2-phenoxyphenyl)- | isocyanate | | |
| | ureido)benzoic acid) | | | |
| 65 | 5,5′-methylenebis(3-chloro- | 4-nitrophenyl isocyanate | I | 78 |
| | 2-(3-(4-nitrophenyl)- | | | |
| | ureido)benzoic acid) | | | |
| 66 | 5,5′-methylenebis(3-chloro- | 3-chlorophenyl | I | 81 |
| | 2-(3-(3-chlorophenyl)- | isocyanate | | |
| | ureido)benzoic acid) | | | |

TABLE 6
Physical properties of the compounds of Examples 48-66

| Example | ¹H NMR (DMSO-d₆, 400 MHz), δ |
|---|---|
| No. | ¹³C NMR (DMSO-d₆, 100 MHz), δ |
| 48 | ¹H; 13.42 (2H, br s), 10.31 (2H, s), 9.96 (2H, s), 8.28 (2H, d), |
| | 7.85-7.76 (2H, m), 7.74-7.72 (2H, m), 7.48-7.42 (2H, m), 7.39-7-34 (2H, m), |
| | 7.29 (2H, t), 7.04-6.98 (2H, m), 3.93 (2H, s). |
| | ¹³C; 169.2, 152.0, 141.3, 140.0, 134.1, 133.8, 133.0, 130.6, 130.2, |
| | 121.5, 120.2, 117.8, 116.8, 115.7, 38.9. |
| 49 | ¹H; 13.36 (2H, br s), 10.22 (2H, s), 9.73 (2H, s), 8.28 (2H, d), |
| | 7.82-7.77 (2H, m), 7.53-7.46 (4H, m), 7.44-7.39 (2H, m), 7.27 (4H, t), 6.97 (2H, |
| | t), 3.92 (2H, s). ¹³C; 168.9, 152.0, 140.1, 139.5, 133.8, 133.4, 130.4, |
| | 128.4 121.8, 120.0, 118.5, 115.4, 38.9. |
| 50 | ¹H; 13.46 (2H, br s), 10.37 (2H, s), 10.12 (2H, s), 8.29 (2H, d), |
| | 8.01-7.98 (2H, m), 7.79-7.83 (2H, m), 7.75-7.69 (2H, m), 7.52-7.39 (6H, m), |
| | 3.94 (2H, s). ¹³C; 169.0, 151.9, 140.5, 139.6, 134.0, 133.8, 130.5, |
| | 129.9, 125.2, 122.9, 120.9, 120.0, 118.6, 115.6, 111.3, 38.9. |
| 51 | ¹H; 13.42, 10.32 (2H, s), 10.00 (2H, s), 8.32 (2H, d), 8.12-8.09 (2H, m), |
| | 7.82-7.76 (4H, m), 7.61-7.56 (2H, m), 7.46-7.40 (4H, m), 3.94 (2H, s), |
| | 2.56 (6H, s). ¹³C; 197.3. 169.0, 152.0, 134.0, 133.8, 133.5, 130.5, |
| | 128.9,, 122.9, 121.8, 120.0, 117.6, 115.4, 38.9, 26.7. |
| 52 | ¹H; 13.33 (2H, br s), 10.20 (2H, s), 9.66 (2H, s), 8.28 (2H, d), |
| | 7.81-7.76 (2H, m), 7.44-7.39 (2H, m), 7.38-7.34 (2H, m), 7.31-7.25 (2H, m), 7.15, |
| | (2H, t), 6.81-6.77 (2H, m), 3.92 (2H, s), 2.27 (6H, s). ¹³C; 168.9, 152.0, |
| | 140.1, 139.4, 137.5, 133.8, 133.3, 128.2, 122.5, 120.0, 119.0, 115.7, |
| | 115.4, 38.9, 21.2. |
| 53 | ¹H; 13.50 (2H, br s), 10.48 (2H, s), 10.45 (2H, s), 8.28 (2H, d), |
| | 8.19 (4H, d), 7.85-7.80 (2H, m), 7.75 (4H, d), 7.50-7.43 (2H, m), 3.95 (2H, |
| | s). ¹³C; 168.9, 151.5, 146.2, 140.9, 139.3, 134.1, 133.9, 130.5, 124.8, |
| | 120.2, 117.6, 116.0, 38.9. |
| 54 | ¹H; 13.28 (2H, br s), 10.17 (2H, s), 8.92 (2H, s), 8.26 (2H, d), |
| | 7.80-7.73 (2H, m), 7.41-7.30 (4H, m), 7.30-7.11 (4H, m), 7.05 (2H, t), 3.90 (2H, |
| | s), 2.23 (6H, s). ¹³C; 168.9, 152.8, 140.2, 136.4, 133.8, 133.2, 131.6, |
| | 130.4, 130.1, 125.8, 124.9, 124.4, 120.0, 115.5, 38.9, 17.9. |
| 55 | ¹H; 13.36 (2H, br s), 10.22 (2H, s), 9.74 (2H, s), 8.27 (2H, d), |
| | 7.82-7.69 (2H, m), 7.46-7.38 (2H, m), 7.23-7.18 (2H, m), 7.17 (2H, t), |
| | 7.08-7.01 (2H, m), 6.59-6.52 (2H, m), 3.92 (2H s), 3.72 (6H, s). ¹³C; 169.0, |
| | 159.3, 151.9, 140.7, 140.o, 133.9, 133.4, 130.4, 129.2, 120.0, 115.4, |
| | 110.7, 107.3, 104.1, 54.7, 38.9. |
| 56 | ¹H; 13.40 (2H, br s), 10.27 (2H, s), 9.88 (2H, s), 8.27 (2H, d), |
| | 7.82-7.78 (2H, m), 7.56-7.49 (4H, m), 7.44-7.39 (2H, m), 7.34-7.29 (4H, m), |
| | 3.92 (2H, s). ¹³C; 169.0, 151.9, 139.9, 138.5, 133.9, 133.5, 130.4, 128.3, |
| | 125.3, 120.0, 119.9, 115.4, 38.9. |
| 57 | 3.92 (s, 2H); 7.37-7.55 (m, 10H); 7.80 (d, J = 1.8 Hz, 2H); 8.27 (d, J = 8.8 Hz, |
| | 2H); 9.92 (s, 2H); 10.30 (s, 2H); 13.0-13.8 (br s, 2H). |
| 58 | 3.76 (s, 2H); 6.68 (d, J = 8.4 Hz, 1H); 7.10 (dd, J = 8.4 and 1.9 Hz, 1H); |
| | 7.33-7.57 (m, 5H); 7.53 (d, J = 1.9 Hz, 1H); 7.74 (d, J = 1.9 Hz, 1H); |
| | 8.1-9.1 (br s, 2H); 8.24 (d, J = 8.6 Hz, 1H); 9.90 (s, 1H); 10.28 (s, 1H); |
| | 12.3-14.1 (br s, 2H). |
| 59 | ¹H; 13.31 (2H, br s), 10.16 (2H, s), 9.53 (2H, s), 8.18 (2H, d), |
| | 7.90-7.83 (2H, m), 7.79-7.75 (2H, m), 7.40-7.36 (2H, m), 7.26-7.19 (2H, m), |
| | 7.17-7.04 (4H, m), 3.92 (2H, s). ¹³C; 168.5, 154.5, 152.2, 139.4, 133.8, |
| | 133.6, 130.4, 126.7, 126.6, 124.1, 123.8, 123.7, 123.3, 120.7, 116.5, |
| | 115.2, 115.0, 93.8, 38.9. |
| 60 | ¹H; 10.49 (1H, s), 10.42 (1H, s), 8.25 (1H, d), 8.21-8.17 (2H, m), |
| | 7.80-7.72 (3H, m), 7.57-7.54 (1H, m), 7.43-7.38 (1H, m), 7.15-7.09 (1H, m), |
| | 6.74 (1H, s), 3.78 (2H, s). ¹³C; 169.1, 169.0, 151.5, 149.5, 146.3, |
| | 140.9, 139.1, 135.0, 134.2, 133.8, 130.5, 130.4, 126.7, 124.8, 120.1, |
| | 117.6, 116.6, 115.8, 109.3, 38.9. |
| 61 | ¹H; 13.28 (2H, br s), 10.20 (2H, s), 9.17 (2H, s), 8.20 (2H, d), |
| | 7.78-7.55 (8H, m), 7.45-7.35 (4H, m), 3.90 (2H, s). |
| 62 | ¹H; 13.4 (2H, br s), 10.42 (2H, s), 9.40 (2H, br s), 8.34 (2H, d), |
| | 7.79-7.75 (2H, m), 7.58-7.7.51 (4H, m), 7.42-7.31 (4H, m), 3.89 (2H, s). ¹³C; |
| | 169.1, 152.1, 140.3, 134.2, 134.1, 133.4, 132.7, 130.5, 128.7, 128.4, |
| | 119.1, 114.7, 38.9. |
| 63 | ¹H; 13.33, (2H, br s), 10.17 (2H, s), 9.50 (2H, s), 8.28 (2H, d), |
| | 7.80-7.75 (2H, m), 7.44-7.33 (6H, m), 6.89-6.82 (4H, m), 3.95-3.87 (6H, m), |
| | 1.71-1.62 (4H, m), 1.48-1.39 (4H, m), 0.93 (6H, t). ¹³C; 169.0, 153.8, |
| | 152.2, 140.4, 134.0, 133.1, 132.3, 120.5, 120.3, 115.1, 114.3, 114.2, |
| | 67.1, 38.9, 30.7, 18.7, 13.6. |
| 64 | ¹H; 13.16 (2H, br s), 9.98 (2H, s), 9.25 (2H, s), 8.07 (2H, d), |
| | 7.99-7.92 (2H, m), 7.74-7.69 (2H, m), 7.41-7.32 (6H, m), 7.15-7.06 (4H, m), |
| | 7.05-6.98 (6H, m), 6.87-6.81 (2H, m), 3.90 (2H, s). ¹³C; 168.3, 156.6, |
| | 152.5, 147.0, 139.2, 133.8, 130.5, 130.4, 129.9, 129.4, 123.6, 123.5, |
| | 123.4, 122.7, 121.4, 118.5, 118.2, 117.1, 38.9. |
| 65 | ¹H; 13.21 (2H, br s), 9.93 (2H, s), 8.75 (2H, br s), 8.22-8.15 (4H, m), |
| | 7.71-7.65 (8H, m), 4.05 (2H, s). ¹³C; 166.9, 151.7, 146.0, 140.8, 138.6, |
| | 132.4, 132.1, 130.1, 129.4, 128.8, 124.9, 117.1, 38.3. |
| 66 | ¹H; 13.15 (2H, br s), 9.40 (2H, s), 8.52 (2H, s), 7.73-7.64 (6H, m), |
| | 7.33-7.19 (4H, m), 7.04-6.80 (2H, m), 4.03 (2H, s). ¹³C; 167.0, 151.9, |
| | 140.9, 138.2, 132.9, 132.4, 132.3, 130.2, 129.7, 129.0, 128.7, 121.3, |
| | 117.1, 116.2, 38.2. |

### General Methods Producing Exemplified Compounds 67-97

### General Method for the Preparation of Examples 67-81 and 83-85

Intermediate VI (250 mg, 0.873 mmol) was added in portions to a 50° C. warm solution of sodium carbonate (466 mg, 2.18 mmol, in 5 mL of water). Arylsulfonyl chloride (2.18 mmol) was added to the solution in portions and the resulting mixture was stirred at 70° C. for 30 min and then at 85° C. for additional 30 min. After cooling to room temperature the reaction mixture was acidified with dilute HCl, the product was collected and washed with dilute HCl and then water to give the title compound as a solid. Recrystallization form an appropriate solvent furnished pure compounds of the Examples as depicted in Table 7.

### Method for Preparation of Example 82

The compound of Example 81 (0.13 g, 0.2 mmol; see below) was dissolved in CH₂Cl₂ (10 mL) and MeOH (1 mL) and Pd/C (0.045 g, 10%) was added. The mixture was set under hydrogen atmosphere and stirred at rt for 1.5 h. Filtration and concentration afforded the crude product which was purified by recrystallization in ethanol/water to furnish the pure title compound in 85 mg (72%) yield.

TABLE 7
Compounds of Examples 67-85

| | | Arylsulfonyl | Yield, |
|---|---|---|---|
| No | Chemical name | chloride | (%) |
| 67 | 5,5′-methylenebis(2-(4-acetamido-3- | 4-acetamido-3- | 45 |
| | chlorophenylsulfonamido)benzoic acid) | chlorobenzene-1- | |
| | | sulfonyl chloride | |
| 68 | 5,5′-methylenebis(2-(4- | 4-nitrobenzene-1- | 78 |
| | nitrophenylsulfonamido)benzoic acid) | sulfonyl chloride | |
| 69 | 5,5′-methylenebis(2-(2- | 2-(trifluoromethyl)benzene- | 67 |
| | (trifluoromethyl)phenylsulfonamido)benzoic | 1- | |
| | acid) | sulfonyl chloride | |
| 70 | 5,5′-methylenebis(2-(4- | 4-cyanobenzene- | 56 |
| | cyanophenylsulfonamido)benzoic acid) | 1-sulfonyl | |
| | | chloride | |
| 71 | 5,5′-methylenebis(2-(3- | 3-(chlorosulfonyl)- | 80 |
| | carboxyphenylsulfonamido)benzoic acid) | benzoic acid | |
| 72 | 5,5′-methylenebis(2-(2-chloro-4- | 2-chloro-4- | 77 |
| | cyanophenylsulfonamido)benzoic acid) | cyanobenzene-1- | |
| | | sulfonyl chloride | |
| 73 | 5,5′-methylenebis(2-(4- | 4-(trifluoromethoxy)benzene- | 51 |
| | (trifluoromethoxy)phenylsulfonamido)benzoic | 1- | |
| | acid) | sulfonyl chloride | |
| 74 | 5,5′-methylenebis(2-(3,5-bis(trifluoromethyl)- | 3,5-bis(trifluoromethyl)benzene- | 86 |
| | phenylsulfonamido)benzoic acid) | 1-sulfonyl | |
| | | chloride | |
| 75 | 5,5′-methylenebis(2-(4-acetamidophenyl- | 4-acetamido | 81 |
| | sulfonamido)benzoic acid) | benzene-1- | |
| | | sulfonyl chloride | |
| 76 | 5,5′-methylenebis(2-(4-butylphenyl- | 4-butylbenzene- | 84 |
| | sulfonamido)benzoic acid) | 1-sulfonyl | |
| | | chloride | |
| 77 | 5,5′-methylenebis(2-(3,4-dichloro- | 3,4- | 76 |
| | phenylsulfonamido)benzoic acid) | dichlorobenzene- | |
| | | 1-sulfonyl | |
| | | chloride | |
| 78 | 5,5′-methylenebis[2-(3,4-dimethoxy- | 3,4-Dimethoxy- | 51 |
| | benzenesulfonylamino)-benzoic acid] | benzenesulfonyl | |
| | | chloride | |
| 79 | 5,5′-methylenebis[2-(naphthalene-2- | naphthalene-2- | 15 |
| | sulfonylamino)-benzoic acid] | sulfonyl chloride | |
| 80 | 5,5′-methylenebis[2-(3-carboxy-4-chloro- | 3-carboxy-4- | 47 |
| | benzenesulfonylamino)-benzoic acid] | chloro- | |
| | | benzenesulfonyl | |
| | | chloride | |
| 81 | 5,5′-methylenebis[2-(2-nitro- | 2-nitro- | 51 |
| | benzenesulfonylamino)-benzoic acid] | benzenesulfonyl | |
| | | chloride | |
| 82 | 5,5′-methylenebis[2-(2-amino- | Reductive | 72 |
| | benzenesulfonylamino)-benzoic acid] | hydrogenation | |
| | | from Example 81 | |
| 83 | 5,5′-methylenebis(2-(4-butoxyphenyl- | 4-butoxyphenyl- | 18 |
| | sulfonamido)benzoic acid) | sulfonyl chloride | |
| 84 | 5,5′-methylenebis(2- | phenylsulfonyl | 70 |
| | (phenylsulfonamido)benzoic acid) | chloride | |
| 85 | 5,5′-methylenebis(2-(4- | 4-fluorophenyl- | 67 |
| | fluorophenylsulfonamido)benzoic acid) | sulfonyl chloride | |

TABLE 8
Physical properties of the compounds of Examples 67-85

| | ¹H NMR (DMSO-d₆, 400 MHz), δ |
|---|---|
| No | ¹³C NMR (DMSO-d₆, 100 MHz), δ |
| 67 | ¹H; 10.95 (2H, br s) 9.67 (2H, s) 8.07 (2H, d, J = 8.6 Hz) 7.82-7.81 (2H, m), |
| | 7.73-7.70 (4H, m) 7.43-7.34 (4H, m) 3.88 (2H, s) 2.14 (6H, s). ¹³C; 169.2, |
| | 169.0, 139.4, 137.3, 136.0, 134.5, 131.2, 127.7, 126.1, 124.7, 124.4, |
| | 119.3, 117.6, 38.5, 23.6. |
| 68 | ¹H; 8.32 (4H, m) 8.02 (4H, m) 7.69-7.67 (2H, m) 7.37-7.28 (4H, m) |
| | 3.84 (2H, s). ¹³C; 168.9, 149.6, 145.1, 138.1, 135.5, 134.1, 131.1, 128.1, 124.4, |
| | 119.4, 118.7, 38.6. |
| 69 | ¹H; 11.32 (2H, br s) 8.24-8.19 (2H, m) 8.20-7.19 (2H, m) 7.90-7.82 (4H, m) |
| | 7.74-7.72 (2H, m) 7.42-7.33 (4H, m) 3.86 (2H, s). ¹³C; 169.2, 137.1, 136.6, |
| | 135.4, 134.6, 134.0, 133.2, 131.4, 131.2, 128.8, 128.7, 117.6, 116.2, 38.4. |
| 70 | ¹H; 11.04 (2H, br s) 8.05-8.00 (4H, m) 7.98-7.92 (4H, m) 7.71-7.68 (2H, m) |
| | 7.39-7.36 (4H, m) 3.89 (2H, s). ¹³C; 168.9, 142.6, 136.6, 136.3, 134.4, |
| | 133.3, 131.1, 127.4, 119.7, 118.1, 117.2, 115.6, 38.5. |
| 71 | ¹H; 10.95 (2H, s) 8.28-8.23 (2H, m) 8.18-8.10 (2H, m) 7.98-7.93 (2H, m) |
| | 7.69-7.61 (4H, m) 7.43-7.33 (4H, m) 3.86 (2H, s). ¹³C; 169.0, 165.4, 138.9, |
| | 137.0, 136.1, 134.5, 133.7, 131.8, 131.1, 130.5, 129.9, 127.0, 119.5, 117.7, 38.5. |
| 72 | ¹H; 11.67 (2H, br s) 8.31-8.24 (4H, m) 8.06-8.01 (2H, m) 7.75-7.73 (2H, m) |
| | 7.33-7.28 (4H, m) 3.84 (2H, s). ¹³C; 169.2, 139.6, 136.5, 135.6, 135.3, |
| | 134.7, 132.1, 131.7, 131.3, 131.1, 117.3, 117.2, 116.5, 116.0, 38.3. |
| 73 | ¹H; 7.93-7.88 (4H, m) 7.72-7.69 (2H, m) 7.54-7.48 (4H, m) 7.41-7.32 (4H, |
| | m) 3.86 (2H, s). ¹³C; 169.0, 151.0, 137.6, 137.5, 135.7, 134.3, 131.1, |
| | 129.3, 121.2, 119.0, 117.7, 38.5. |
| 74 | ¹H; 10.79 (2H, br s) 8.45-8.40 (2H, m) 8.23-8.20 (4H, m) 7.70-7.68 (2H, m) |
| | 7.39-7.34 (4H, m) 3.91 (2H, s). |
| 75 | ¹H; 10.85 (2H, s) 10.32 (2H, s) 7.74-7.67 (10H, m) 7.42-7.31 (4H, m) |
| | 3.84 (2H s) 2.06 (6H, s). ¹³C; 169.2, 168.8, 143.4, 137.8, 135.4, 134.5, 131.7, |
| | 131.0, 127.9, 118.6, 118.4, 116.6, 38.5, 24.0. |
| 76 | ¹H; 7.68-7.62 (6H, m), 7.40-7.25 (8H, m) 3.80 (2H, s) 2.63-2.55 (4H, m) |
| | 1.55-1.45 (4H, m) 1.30-1.18 (4H, m) 0.84 (6H, t, J = 7.4 Hz). ¹³C; 169.2, |
| | 148.0, 138.4, 135.1, 133.9, 130.9, 128.9, 127.1, 126.5, 125.2, 118.3, 38.6, |
| | 34.4, 32.4, 21.6, 13.6. |
| 77 | ¹H; 10.99 (2H, br s), 7.97-7.94 (2H, m) 7.82-7.78 (2H, m) 7.74-7.68 (4H, m) |
| | 7.39-7.36 (4H, m) 3.90 (2H, s). ¹³C; 168.8, 138.9, 136.6, 136.4, 136.3, |
| | 134.3, 132.1, 131.5, 131.1, 128.4, 126.6, 120.0, 118.4, 38.6. |
| 78 | ¹H; 3.67 (s, 6H) 3.78 (s, 6H) 3.86 (s, 2H) 7.04 (d, J = 8.5 Hz, 2H) 7.15 (d, |
| | J = 2.1 Hz, 2H) 7.31-7.50 (m, 6H) 7.71 (d, J = 1.8 Hz, 2H) 10.75-10.85 (br s, 2H) |
| 79 | ¹H; 3.76 (s, 2H) 7.27 (d, J = 8.6 Hz, 2H) 7.44 (d, J = 8.6 Hz, 2H) 7.57-7.77 (m, |
| | 8H) 7.93-8.17 (m, 6H) 8.52 (s, 2H) 10.8-11.2 (br s, 2H) |
| 80 | ¹H; 3.86 (s, 2H) 7.29-7.40 (m, 4H); 7.66-7.76 (m, 4H); 7.85 (dd, J = 8.4 and |
| | 2.1 Hz, 2H); 8.13 (d, J = 2.1 Hz, 2H) |
| 81 | ¹H; 3.85 (s, 2H) 7.35 (dd, J = 8.6 and 1.8 Hz, 2H) 7.47 (d, J = 8.6 Hz, 2H) |
| | 7.69-7.91 (m, 6H) 7.99 (dd, J = 7.6 and 1.3 Hz, 2H) 8.11 (dd, J = 7.4 and 1.5 Hz, |
| | 2H) 11.3-11.6 (br s, 2H) |
| 82 | ¹H; 3.81 (s, 2H) 5.8-6.2 (br s, 4H) 6.51-6.62 (m, 2H) 6.78 (d, J = 8.2 Hz, 2H) |
| | 7.18-7.36 (m, 6H) 7.53 (d, J = 8.2 Hz, 2H) 7.89 (s, 2H) 11.0-11.6 (br s, 2H) |
| 83 | ¹H; 0.93 (t, J = 7.3 Hz, 6H) 1.35-1.50 (m, 4H) 1.65-1.80 (m, 4H) 3.81 (s, 2H) |
| | 3.90-3.97 (m, 4H) 6.80-6.91 (m, 4H) 7.20-7.24 (m, 2H) 7.56-7.61 (m, 2H) |
| | 7.66-7.71 (m, 2H) 7.72-7.79 (m, 4H) 10.27 (s, 2H) |
| 84 | ¹H; 8.64-8.54 (2H, m) 7.58-7.20 (14H, m) 3.81 (2H, s) |
| 85 | ¹H; 3.87 (s, 2H) 7.33-7.44 (m, 8H) 7.68-7.71 (m, 2H) 7.81-7.88 (m, 4H) |
| | 11.0 (br s, 2H) |

### Method for Benzylation Furnishing Examples 86-95

Compound IV (160 mg, 0.5 mmol) was dissolved in an appropriate solvent (acetonitrile, dichloromethane or ethanol). Benzaldehyde (2 mmol) and NaBH(OAc)₃ (850 mg, 4 mmol) were added and the resulting mixture was stirred at rt for 40-60 h. Extractive workup (CH₂Cl₂, water), drying (Na₂SO₄) of the combined organic extracts and concentration furnished the crude which was purified by chromatography and then hydrolyzed according to general method (e.g. as described in general method A) to give the pure compounds as depicted in Table 9.

TABLE 9
Compounds of Examples 86-95

| | | | Yield (%) | |
|---|---|---|---|---|
| No | Chemical name | Benzaldehyde | Ester | Acid |
| 86 | 5-(3-Carboxy-4-(4-fluoro-benzylamino)- | 4-fluoro- | 72 | 72 |
| | benzyl)-2-(4-fluoro-benzylamino)-benzoic acid | benzaldehyde | | |
| 87 | 5-(3-Carboxy-4-(3-trifluoromethyl- | 3-trifluoro- | 70 | 53 |
| | benzylamino)-benzyl)-2-(3- | methyl- | | |
| | trifluoromethyl-benzylamino)-benzoic acid | benzaldehyde | | |
| 88 | 5-(3-Carboxy-4-(4-methoxy- | 4-methoxy- | 65 | 75 |
| | benzylamino)-benzyl)-2-(4-methoxy- | benzaldehyde | | |
| | benzylamino)-benzoic acid | | | |
| 89 | 5-(3-Carboxy-4-(4-methyl- | 4-methyl- | 75 | 63 |
| | benzylamino)-benzyl)-2-(4-methyl- | benzaldehyde | | |
| | benzylamino)-benzoic acid | | | |
| 90 | 5-(3-Carboxy-4-(4-chloro-benzylamino)- | 4-chloro- | 59 | 74 |
| | benzyl)-2-(4-chloro-benzylamino)-benzoic acid | benzaldehyde | | |
| 91 | 2-Benzylamino 5-(3-carboxy-4- | benzaldehyde | 46 | 71 |
| | (benzylamino)-benzyl)-)-benzoic acid | | | |
| 92 | 2-(4-Benzyloxy)-benzylamino 5-(3- | 4-benzyloxy- | 65 | 85 |
| | carboxy-4-(4-benzyloxy)-benzylamino)- | benzaldehyde | | |
| | benzyl)-)-benzoic acid | | | |
| 93 | 2-(3-Benzyloxy)-benzylamino)-5-(3-carboxy- | 3-benzyloxy- | 65 | 62 |
| | 4-(3-benzyloxy)-benzylamino)-benzyl)-benzoic acid | benzaldehyde | | |
| 94 | 5-(3-Carboxy-4-(2,3-dichloro- | 2,3-dichloro- | 62 | 71 |
| | benzylamino)-benzyl)-2-(2,3-dichloro- | benzaldehyde | | |
| | benzylamino)-benzoic acid | | | |
| 95 | 5-(3-Carboxy-4-(3,5-dichloro- | 3,5-dichloro- | 58 | 77 |
| | benzylamino)-benzyl)-2-(3,5-dichloro- | benzaldehyde | | |
| | benzylamino)-benzoic acid | | | |

TABLE 10
Physical properties of the compounds of Examples 86-95

| No | ¹H NMR (DMSO-d₆, 400 MHz), δ |
|---|---|
| 86 | 7.60 (2H, d, J = 2.1 Hz) 7.41-7.29 (4H, m) 7.21-7.07 (6H, m) 6.60 (2H, d, |
| | J = 8.7 Hz) 4.40 (4H, s) 3.64 (2H, s) |
| 87 | 7.69-7.50 (10H, m) 7.12 (2H, dd, J = 8.7 and 2.1 Hz) 6.55 (2H, d, J = 8.7 Hz) |
| | 4.54 (4H, s) 3.64 (2H, s) |
| 88 | 7.60 (2H, d, J = 2.0 Hz) 7.30-7.20 (4H, m) 7.13 (2H, dd, J = 8.67 and 2.0 Hz) |
| | 6.94-9.83 (4H, m) 6.62 (2H, d, J = 8.7 Hz) 4.30 (4H, s) 3.72 (6H, s) |
| | 3.64 (2H, s) |
| 89 | 7.59 (2H, d, J = 2.1 Hz) 7.24-7.07 (10H, m) 6.59 (2H, d, J = 8.7 Hz) 4.35 (4H, |
| | s) 3.64 (2H, s) 2.26 (6H, s) |
| 90 | 7.60 (2H, d, J = 2.1 Hz) 7.42-7.28 (8H, m) 7.12 (2H, dd, J = 8.7 and 2.1 Hz) |
| | 6.54 (2H, d, J = 8.7 Hz) 4.43 (4H, s) 3.63 (2H, s) |
| 91 | 7.60 (2H, d, J = 2.1 Hz) 7.38-7.18 (10H, m) 7.12 (2H, dd, J = 8.6 and 2.1 Hz) |
| | 6.59 (2H, d, J = 8.6 Hz) 4.44 (4H, s) 3.63 (2H, s) |
| 92 | 7.59 (2H, d, J = 2.0 Hz) 7.48-7.20 (14H, m) 7.13 (2H, dd, J = 8.6 and 2.0 Hz) |
| | 7.01-6.92 (4H, m) 6.62 (2H, d, J = 8.6 Hz) 5.06 (4H, s) 4.32 (4H, s) 3.64 (2H, s) |
| 93 | 7.66 (2H, d, J = 2.0 Hz) 7.42-7.12 (12H, m) 7.07 (2H, dd, J = 8.6 and 2.0 Hz) |
| | 6.97-6.78 (6H, m) 6.56 (2H, d, J = 8.6 Hz) 5.06 (4H, s) 4.42 (4H, s) 3.64 (2H, s) |
| 94 | 8.4-8.1 (2H, br s) 7.63 (2H, d, J = 1.5 Hz) 7.53 (2H, dd, J = 6.7 and 2.7 Hz) |
| | 7.35-7.23 (4H, m) 7.13 (2H, dd, J = 8.5 and 1.5 Hz) 6.47 (2H, d, J = 8.5 Hz) |
| | 4.54 (4H, s) 3.65 (2H, s) |
| 95 | 8.7-7.8 (2H, br s) 7.62 (2H, d, J = 1.9 Hz) 7.48-7.42 (2H, m) 7.34 (4H, d, |
| | J = 1.6 Hz) 7.13 (2H, dd, J = 8.6 and 1.9 Hz) 6.52 (2H, d, J = 8.6 Hz) 4.47 (4H, s) |
| | 3.65 (2H, s) |

### Method for the Preparation of Examples 96 and 97

Step 1: To a solution of intermediate VI (523 mg, 1.83 mmol) in THF was added 4-nitrobenzeneisocyanate (100 mg, 0.610 mmol) and the resulting solution stirred at room temperature overnight. n-Hexane was added, the precipitated product was collected and washed with diluted HCl (aq) to give 2-amino-5-(3-carboxy-4-(3-(4-nitrophenyl)ureido)benzyl)benzoic acid (Example 60 above) as a yellow solid (185 mg, 67%).

Step 2: The foregoing compound of Example 60 (250 mg, 0.555 mmol) was treated with arylsulfonyl chloride (0.666 mmol) and sodium carbonate (235 mg, 2.22 mmol, in 5 mL of water) as described above. The crude product was purified by chromatography to give the pure compounds described in Table 11.

TABLE 11
Compounds of Examples 96-97

| | | | Yield |
|---|---|---|---|
| No | Chemical name | Aryl sulfonyl chloride | (%) |
| 96 | 5-(3-carboxy-4-(3,4- | 3,4- | 50 |
| | dichlorophenylsulfonamido)benzyl)- | dichlorophenylsulfonyl | |
| | (2-3-(4-nitrophenyl)ureido)benzoic | chloride | |
| | acid | | |
| 97 | 5-(3-carboxy-4-(3-(4- | 4-nitrophenylsulfonyl | 60 |
| | nitrophenyl)ureido)benzyl)-2-(4- | chloride | |
| | nitrophenylsulfonamido)benzoic acid | | |

TABLE 12
Physical properties of the compounds of Examples 96-97

| | ¹H NMR (DMSO-d₆, 400 MHz), δ |
|---|---|
| No | ¹³C NMR (DMSO-d₆, 100 MHz), δ |
| 96 | ¹H 10.48 (1H, s) 10.43 (1H, s) 8.29-8.24 (1H, m) 8.22-8.16 (2H, m) 7.99-7.97 (1H, |
| | m), 7.85-7.69 (6H, m) 7.47-7.38 (3H, m) 3.93 (2H, s). |
| | ¹³C 168.9, 151.5, 146.2, 140.9, 139.4, 136.8, 136.2, 134.3, 133.9, 133.7, 132.1, |
| | 131.6, 131.0, 130.6, 128.4, 126.6, 124.8, 120.2, 120.0, 118.5, 117.6, 115.9, 38.7. |
| 97 | ¹H 10.48 (1H, s) 10.42 (1H, s) 8.36-8.31 (2H, m) 8.27-8.23 (1H, m) 8.22-8.17 (2H, |
| | m) 8.07-8.02 (2H, m) 7.79-7.72 (4H, m) 7.42-7.39 (3H, m) 3.91 (2H, s). |
| | ¹³C 168.9, 151.5, 149.7, 146.2, 140.9, 139.4, 136.6, 134.3, 134.0, 133.7, 131.1, |
| | 130.6, 128.2, 124.8, 124.5, 120.2, 119.6, 117.6, 115.9, 38.7. |

### Example 98

Title compounds of the examples were tested in the biological test described above and were found to exhibit 50% inhibition of LTC₄ at a concentration of 10 μM or below. For example, the following representative compounds of the examples exhibited the following IC₅₀ values.

### Example 6: 5700 nM

### Example 8: 740 nM

### Example 9: 3400 nM

### Example 12: 2800 nM

### Example 27: 6410 nM

### Example 55: 1800 nM

### Example 57: 4000 nM

### Example 58: 870 nM

### Example 59: 4300 nM

### Example 62: 5800 nM

### Example 67: 2800 nM

### Example 69: 1300 nM

### Example 74: 700 nM

### Example 89: 3400 nM

Example 93: 2300 nM

1. A compound of formula I, 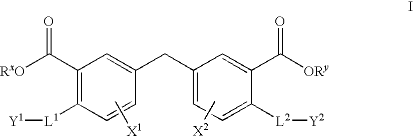
wherein
Y¹ represents H or —Ar¹;
Y² represents H or —Ar²;
wherein at least one of Y¹ and Y² is other than H;
X¹ and X² each independently represent one or more optional substituents selected from halo, —R^{3a}, —CN, —C(O)R^{3b}, —C(O)OR^{3c}, —C(O)N(R^{4a})R^{5a}, —N(R^{4b})R^{5b}, —N(R^{3d})C(O)R^{4c}, —N(R^{3e})C(O)N(R^{4d})R^{5d}, —N(R^{3f})C(O)OR^{4e}, —N₃, —NO₂, —N(R^{3g})S(O)₂N(R^{4f})R^{5f}, —OR^{3b}, —OC(O)N(R^{4g})R^{5g}, —OS(O)₂R³ⁱ, —S(O)ₘR^{3j}, —N(R^{3k})S(O)₂R^{3m}, —OC(O)R³ⁿ, —OC(O)OR^{3p}, —S(O)₂N(R^{4b})R^{5h} or —OS(O)₂N(R⁴ⁱ)R⁵ⁱ;
m represents 0, 1 or 2;
R^{3b} to R^{3h}, R^{3j}, R^{3k}, R³ⁿ, R^{4a} to R⁴ⁱ, R^{5a}, R^{5b}, R^{5d} and R^{5f} to R⁵ⁱ each independently represent H or R^{3a}; or
any of the pairs R^{4a} and R^{5a}, R^{4b} and R^{5b}, R^{4d} and R^{5d}, R^{4f} and R^{5f}, R^{4g} and R^{5g}, R^{4h} and R^{5h} or R⁴ⁱ and R⁵ⁱ may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or R^{3a};
R³ⁱ, R^{3m} and R^{3p} each independently represent R^{3a};
R^{3a} represents C₁₋₆ alkyl optionally substituted by one or more substituents selected from F, Cl, —CN, —N₃, ═O, —OR^{6a}, —N(R^{6b})R^{7b}, —S(O)ₙR^{6c}, —S(O)₂N(R^{6d})R^{7d} or —OS(O)₂N(R^{6e})R^{7e};
n represents 0, 1 or 2;
R^{6a}, R^{6b}, R^{6c}, R^{6d} and R^{6e} each independently represent H or C₁₋₆ alkyl optionally substituted by one or more substituents selected from F, Cl, ═O, —OR^{8a}, —N(R^{9a})R^{10a} or —S(O)₂-M¹;
R^{7b}, R^{7d} and R^{7e} each independently represent H, —S(O)₂CH₃, —S(O)₂CF₃ or C₁₋₆ alkyl optionally substituted by one or more substituents selected from F, Cl, ═O, —OR^{11a}, —N(R^{12a})R^{13a} or —S(O)₂-M²; or
R^{6b} and R^{7b}, R^{6d} and R^{7d} or R^{6e} and R^{7e} may be linked together to form a 3- to 6-membered ring, which ring optionally contains a further heteroatom (such as nitrogen or oxygen) in addition to the nitrogen atom to which these substituents are necessarily attached, and which ring is optionally substituted by F, Cl, ═O or C₁₋₃ alkyl optionally substituted by one or more substituents selected from ═O and fluoro;
M¹ and M² each independently represent —CH₃, —CH₂CH₃, —CF₃ or —N(R^{14a})R^{15a};
R^{8a} and R^{11a} each independently represent H, —CH₃, —CH₂CH₃, —CF₃ or —CHF₂;
R^{9a}, R^{10a}, R^{12a}, R^{13a}, R^{14a} and R^{15a} each independently represent H, —CH₃ or —CH₂CH₃,
Ar¹ and Ar² each independently represent an aryl group or a heteroaryl group, both of which groups are optionally substituted by one or more substituents selected from A;
A represents:
I) an aryl group or a heteroaryl group, both of which are optionally substituted by one or more substituents selected from B;
II) C₁₋₈ alkyl or a heterocycloalkyl group, both of which are optionally substituted by one or more substituents selected from G¹ and/or Z¹; or
III) a G¹ group;
G¹ represents halo, cyano, —N₃, —NO₂, —ONO₂ or -A¹R^{16a};
wherein A¹ represents a single bond or a spacer group selected from —C(O)A²-, —S—, —S(O)₂A³-, —N(R^{17a})A⁴- or —OA⁵-, in which:
A² represents a single bond, —O—, —N(R^{17b})— or —C(O)—;
A³ represents a single bond, —O— or —N(R^{17c})—;
A⁴ and A⁵ each independently represent a single bond, —C(O)—, —C(O)N(R^{17d})—, —C(O)O—, —S(O)₂— or —S(O)₂N(R^{17e})—;
Z¹ represents ═O, ═S, ═NOR^{16b}, ═NS(O)₂N(R^{17f})R^{16c}, ═NCN or ═C(H)NO₂;
B represents:
I) an aryl group or a heteroaryl group, both of which are optionally substituted by one or more substituents selected from G²;
II) O₁₋₈ alkyl or a heterocycloalkyl group, both of which are optionally substituted by one or more substituents selected from G² and/or Z²; or
III) a G² group;
G² represents halo, cyano, —N₃, —NO₂, —ONO₂ or -A⁶-R^{18a};
wherein A⁶ represents a single bond or a spacer group selected from —C(O)A⁷-, —S—, —S(O)₂A⁸-, —N(R^{19a})A⁹- or —OA¹⁰-, in which:
A⁷ represents a single bond, —O—, —N(R^{19b})— or —C(O)—;
A⁸ represents a single bond, —O— or —N(R^{19c})—;
A⁹ and A¹⁰ each independently represent a single bond, —C(O)—, —C(O)N(R^{19d})—, —C(O)O—, —S(O)₂— or —S(O)₂N(R^{19e})—;
Z² represents ═O, ═S, ═NOR^{18b}, ═NS(O)₂N(R^{19f})R^{18c}, ═NCN or ═C(H)NO₂;
R^{16a}, R^{16b}, R^{16c}, R^{17a}, R^{17b}, R^{17c}, R^{17d}, R^{17e}, R^{17f}, R^{18a}, R^{18b}, R^{18c}, R^{19a}, R^{19b}, R^{19c}, R^{19d}, R^{19e} and R^{19f} are each independently selected from:
i) hydrogen;
ii) an aryl group or a heteroaryl group, both of which are optionally substituted by one or more substituents selected from G³;
iii) C₁₋₈ alkyl or a heterocycloalkyl group, both of which are optionally substituted by one or more substituents selected from G³ and/or Z³; or
any pair of R^{16a} to R^{16c} and R^{17a} to R^{17f}, and/or R^{18a} to R^{18c} and R^{19a} to R^{19f}, may, be linked together to form with those, or other relevant, atoms a further 3- to 8-membered ring, optionally containing 1 to 3 heteroatoms and/or 1 to 3 double bonds, which ring is optionally substituted by one or more substituents selected from G³ and/or Z³;
G³ represents halo, cyano, —N₃, —NO₂, —ONO₂ or -A¹¹-R^{20a};
wherein A¹¹ represents a single bond or a spacer group selected from —C(O)A¹²-, —S—, —S(O)₂A¹³-, —N(R^{21a})A¹⁴- or —OA¹⁵-, in which:
A¹² represents a single bond, —O—, —N(R^{21b})— or —C(O)—;
A¹³ represents a single bond, —O— or —N(R^{21c})—;
A¹⁴ and A¹⁵ each independently represent a single bond, —C(O)—, —C(O)N(R^{21d})—, —C(O)O—, —S(O)₂— or —S(O)₂N(R^{21e})—;
Z³ represents ═O, ═S, ═NOR^{20b}, ═NS(O)₂N(R^{21f})R^{20c}, ═NCN or ═C(H)NO₂;
R^{20a}, R^{20b}, R^{20c}, R^{21a}, R^{21b}, R^{21c}, R^{21d}, R^{21e} and R^{21f} are each independently selected from:
i) hydrogen;
ii) C₁₋₆ alkyl or a heterocycloalkyl group, both of which groups are optionally substituted by one or more substituents selected from halo, C₁₋₄ alkyl, —N(R^{22a})R^{23a}, —OR^{22b} and ═O; and
iii) an aryl or heteroaryl group, both of which are optionally substituted by one or more substituents selected from halo, C₁₋₄ alkyl (optionally substituted by one or more substituents selected from ═O, fluoro and chloro), —N(R^{22c})R^{23b} and —OR^{22d}; or
any pair of R^{20a} to R^{20c} and R^{21a} to R^{21f} may be linked together to form with those, or other relevant, atoms a further 3- to 8-membered ring, optionally containing 1 to 3 heteroatoms and/or 1 or 2 double bonds, which ring is optionally substituted by one or more substituents selected from halo, C₁₋₄ alkyl, —N(R^{22e})R^{23c}, —OR^{22f} and ═O;
L¹ represents —N(R^{w})A¹⁹-;
L² represents —N(R^{z})A²⁰-;
A¹⁹ represents a single bond, —C(O)N(R^{w})—, —S(O)₂— or —CH₂—;
A²⁰ represents a single bond, —C(O)N(R^{z})—, —S(O)₂— or —CH₂—;
but wherein when A¹⁹ represents —S(O)₂—, Y¹ represents Ar¹, and when A²⁰ represents —S(O)₂—, then Y² represents Ar²;
R^{x}, R^{y}, R^{w} and R^{z} each independently represent H, C₁₋₁₄ alkyl (optionally substituted by one or more substituents selected from halo, —CN, —N(R^{24a})R^{25a}, —OR^{24b}, ═O, aryl and heteroaryl (which latter two groups are optionally substituted by one or more substituents selected from halo, C₁₋₄ alkyl (optionally substituted by one or more substituents selected from fluoro, chloro and ═O), —N(R^{24c})R^{25b} and —OR^{24d}));
R^{22a}, R^{22b}, R^{22c}, R^{22d}, R^{22e}, R^{22f}, R^{23a}, R^{23b}, R^{23c}, R^{24a}, R^{24b}, R^{24c}, R^{24d}, R^{25a} and R^{25b} are each independently selected from hydrogen and C₁₋₄ alkyl, which latter group is optionally substituted by one or more substituents selected from fluoro, chloro or ═O,
or a pharmaceutically-acceptable salt thereof, provided that, when X¹ and X² are not present, and:
(a) R^{x} and R^{y} each independently represent H or methyl and L¹ and L² both represent —N(H)—CH₂—, then Ar¹ and Ar² do not both represent unsubstituted phenyl;
(b) R^{x} and R^{y} each independently represent H or methyl optionally substituted by unsubstituted phenyl, or one of R^{x} and R^{y} represents H and the other represents methyl, and L¹ and L² both represent —N(H)—S(O)₂—, then Ar¹ and Ar² do not both represent 4-methylphenyl; and
(c) when R^{x} and R^{y} both represent H, and L¹ and L² both represent —N(H)—S(O)₂, then Ar¹ and Ar² do not both represent 1-hydroxynaphthyl.

2. The compound according to claim 1, wherein A represents G¹ or C₁₋₄ alkyl optionally substituted by one or more G¹ substituents.

3. The compound according to claim 1, wherein G¹ represents halo, cyano, —NO₂ or -A¹-R^{16a}.

4. The compound according to claim 1, wherein G¹ represents halo or -A¹-R^{16a}.

5. The compound according to claim 3, wherein A¹ represents —C(O)A² or —OA⁵.

6. The compound according to claim 1, wherein A² represents —O— or a single bond.

7. The compound according to claim 1, wherein A⁵ represents a single bond.

8. The compound according to claim 1, wherein R^{16a} represents H or C₁₋₆ alkyl optionally substituted by one or more fluoro substituents.

9. The compound according to claim 1, wherein R^{16a} represents C₁₋₄ alkyl or an aryl or heteroaryl group, which latter two are optionally substituted by one or more G³ groups, in which G³ represents halo or A¹¹-R^{20a}.

10. The compound according to claim 1, wherein X¹ and X² each independently represent halo or is/are not present.

11. The compound according to claim 1, wherein R^{x} and R^{y} each independently represent H.

12. The compound according to claim 1, wherein R^{w} and R^{z} each independently represent H or C₁₋₂ alkyl.

13. The compound according to claim 1, wherein Ar¹ and Ar² represent an optionally substituted phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl or benzodioxanyl, group.

14. The compound according to claim 13, wherein Ar¹ and Ar² each independently represent optionally substituted thienyl, thiazolyl, pyridyl, phenyl or naphthyl.

15. The compound according to claim 13 or claim 14, wherein the optional substituents are selected from halo; cyano; —NO₂; C₁₋₆ alkyl optionally substituted with one or more halo groups; heterocycloalkyl optionally substituted by one or more substituents selected from C₁₋₃ alkyl and ═O; —OR²⁶; —C(O)OR²⁶, —C(O)R²⁶ and —N(R²⁶)R²⁷, wherein R²⁶ and R²⁷ independently represent H or C₁₋₆ alkyl optionally substituted by one or more halo groups or aryl optionally substituted by one or more halo or C₁-C₃ alkyl groups (which latter is optionally substituted by one or more halo atoms).

16. The compound according to claim 1, wherein, when Ar¹ and Ar² are substituted with one or two substituents.

17. The compound according to claim 16, wherein Ar¹ and Ar² are the same.

18. The compound according to claim 1, wherein A¹⁹ represents a single bond or —C(O)N(R^{w})— and A²⁰ represents —C(O)N(R^{z})—.

19. The compound according to claim 18 wherein R^{w} and R^{z} are both H.

20. The compound according to claim 1, wherein where A¹⁹ and A²⁰ both represent single bonds.

21. A compound of formula I as defined in claim 1, without proviso (c), or a pharmaceutically acceptable salt thereof, for use as a pharmaceutical.

22. A pharmaceutical formulation including a compound of formula I, as defined in claim 1, without proviso (c), or a pharmaceutically acceptable salt thereof, in admixture with a pharmaceutically acceptable adjuvant, diluent or carrier.

23. A compound of formula I as defined in claim 1, without provisos (a) to (c), or a pharmaceutically acceptable salt thereof, for use in the treatment of a disease by inhibition of the synthesis of leukotriene C₄.

24. Use of a compound of formula I, as defined in claim 1, without provisos (a) to (c), or a pharmaceutically acceptable salt thereof, for the manufacture of a medicament for the treatment of a disease in which inhibition of the synthesis of leukotriene C₄.

25. The compound according to claim 23 or a use according to claim 24, wherein the disease is a respiratory disease, inflammation and/or has an inflammatory component.

26. The compound or the use according to claim 25 wherein the disease is an allergic disorder, asthma, childhood wheezing, a chronic obstructive pulmonary disease, bronchopulmonary dysplasia, cystic fibrosis, an interstitial lung disease, an ear, a nose or a throat disease, an eye disease, a skin disease, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anaemia, hypereosinofilic syndrome, or a malignancy.

27. The use according to claim 26, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain.

28. A method of treatmenting a disease by inhibiting the synthesis of leukotriene C₄, the method comprising administration of a therapeutically effective amount of a compound of formula I as defined in claim 1, without provisos (a) to (c), or a pharmaceutically-acceptable salt thereof, to a patient suffering from, or susceptible to, such a disease.

29. A combination product comprising:
(A) a compound of formula I as defined in claim 1, without provisos (a) to (c), or a pharmaceutically-acceptable salt thereof; and
(B) another therapeutic agent that is useful in treating a respiratory disorder and/or inflammation,
wherein each of components (A) and (B) is formulated in admixture with a pharmaceutically-acceptable adjuvant, diluent or carrier.

30. The combination product according to claim 29 wherein components (A) and (B) are formulated in a single composition with a pharmaceutically-acceptable adjuvant, diluent or carrier.

31. A kit comprising:
(a) a pharmaceutical formulation including a compound of formula I as defined in claim 1, without provisos (a) to (c), or a pharmaceutically-acceptable salt thereof, in admixture with a pharmaceutically-acceptable adjuvant, diluent or carrier; and
(b) a pharmaceutical formulation including another therapeutic agent that is useful in treating a respiratory disorder and/or inflammation in admixture with a pharmaceutically-acceptable adjuvant, diluent or carrier,
wherein components (a) and (b) are each provided in a form that is suitable for administration in conjunction with each other.

32. A process for the preparation of a compound according to claim 1, the process comprising:
(i) for compounds of formula I in which A¹⁹ and A²⁰ represent a single bond, reaction of a compound of formula II, 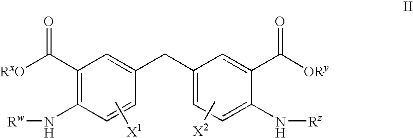
or a protected derivative thereof, wherein R^{x} and R^{y} are as defined in claim 1 and X¹, X², R^{w} and R^{z} are as defined in claim 1, with a compound of formula III,
Ar^{a}-L^{a} III
wherein Ar^{a} represents Ar¹ or Ar² (as appropriate) and L^{a} represents a suitable leaving group;
(ii) for compounds of formula I in which R^{w} and/or R^{z} do not represent hydrogen, reaction of a corresponding compound of formula I in which R^{w} or R^{z} (as appropriate) do represent hydrogen with a compound of formula IV,
R^{wz}-L^{b} IV
wherein R^{wz} represents either R^{w} or R^{z} (as appropriate) as defined in claim 1 provided that it/they does/do not represent hydrogen, and L^{b} represents a suitable leaving group;
(iii) for compounds of formula I that contain only saturated alkyl groups, reduction of a corresponding compound of formula I that contains an unsaturation;
(iv) for compounds of formula I that contain amine groups, reduction of a corresponding compound of formula I that contains a group that may be reduced to an amine group;
(v) for compounds of formula I in which A¹⁹ and A²⁰ independently represent a single bond or —CH₂—, and R^{w} or R^{z} represents optionally substituted C₁₋₁₄ alkyl, reductive amination of a compound of formula I in which R^{w} and/or R^{z} represents H, with a compound of formula V,
R^{wz1}═O V
wherein R^{wz1} represents C₁₋₁₃ alkyl optionally substituted with the substituents as defined in claim 1 in respect of R^{w} or R^{z};
(vi) reaction of a compound of formula VI, 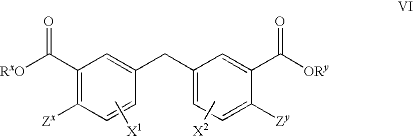
wherein Z^{x} and Z^{y} independently represent a suitable leaving group, and R^{x}, R^{y}, X¹ and X² are as defined in claim 1, with a compound of formula VII,
Y^{a}-A²¹-NH₂ VII
wherein Y^{a} represents Ar¹ or Ar² (as appropriate) as defined in claim 1, A²¹ represents A¹⁹ or A²⁰ (as appropriate);
(vii) for compounds of formula I in which A¹⁹ and/or A²⁰ represents —CH₂—, reductive amination of a compound of formula II as defined above, in the presence of a compound of formula VIII,
Ar^{a}CH═O VIII
wherein Ar^{a} is as defined in claim 1;
(viii) for compounds of formula I in which A¹⁹ and/or A²⁰ represents —CH₂—, reaction of a compound of formula II as defined above, with a compound of formula IX,
Ar^{a}C(O)Cl IX
wherein Ar^{a} is as defined in claim 1;
(ix) for compounds of formula I in which R^{x} and R^{y} represent hydrogen, hydrolysis of a corresponding compound of formula in which R^{x} and R^{y} do not represent hydrogen, or other carboxylic acid or ester protected derivatives thereof;
(x) for compounds of formula I in which R^{x} and R^{y} do not represent hydrogen, esterification of a corresponding compound of formula I in which R^{x} and R^{y} represent hydrogen (or trans-esterification of a compound of formula I in which R^{x} and R^{y} do not represent hydrogen or the same value of the corresponding R^{x} and R^{y} groups in the compound of formula I to be prepared), in the presence of a compound of formula X,
R^{b}—OH X
wherein R^{b} represents R^{x} or R^{y} (as appropriate) provided that it does not represent hydrogen;
(xi) for compounds of formula I in which A¹⁹ and A²⁰ represent —S(O)₂— or —CH₂—, reaction of a compound of formula II as defined above, with a compound of formula XI,
Y^{a}-A^{x}-L^{c} XI
wherein Y^{a} is as defined above, L^{c} represents a suitable leaving group and A^{x} represents either —CH₂— or —S(O)₂—;
(xii) for the preparation of compounds of formula I in which A¹⁹ and A²⁰ both represent —C(O)N(H)—, reaction of a compound of formula II as defined above, or a protected (e.g. at one of the amino groups) derivative thereof, with either:
(A) a compound of formula XII,
Y^{a}—N═C═O XII
or (B) with CO (or a reagent that is a suitable source of CO (e.g. Mo(CO)₆ or Co₂(CO)₈)) in the presence of a compound of formula XIII,
Y^{a}—NH₂ XIII
wherein, in both cases, Y^{a} is as defined above;
(xiii) for the preparation of compounds of formula I in which A¹⁹ and A²⁰ both represent —C(O)N(H)—, reaction of a compound of formula XIV, 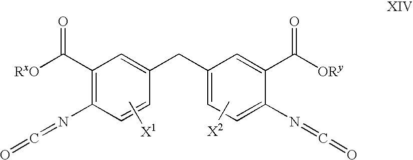
wherein R^{x}, R^{y}, X¹ and X² are as defined in claim 1, with a compound of formula XIII as defined above; or
(xiv) reaction of a compound of formula XV (or two different compounds of formula XV for preparation of compounds of formula I in which R^{x} and R^{y}, X¹ and X² and/or Y¹ and Y² are different), 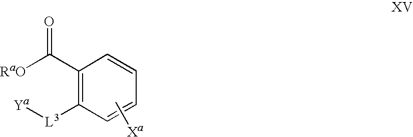
wherein R^{a} represents R^{x} or R^{y} (as appropriate and in which these substituents are preferably other than hydrogen and are preferably the same), L³ represents L¹ or L² (as appropriate), X^{a} represents X¹ or X² (as appropriate), Y^{a} is as defined above, with formaldehyde.

33. The process for the preparation of a pharmaceutical formulation, the process comprising bringing into association a compound of formula I, as defined in claim 1, without proviso (c), or a pharmaceutically acceptable salt thereof with a pharmaceutically-acceptable adjuvant, diluent or carrier.

34. The A process for the preparation of a combination product, the process comprising bringing into association a compound of formula I, as defined in claim 1, without provisos (a) to (c), or a pharmaceutically acceptable salt thereof, with the other therapeutic agent that is useful in the treatment of a respiratory disorder and/or inflammation, and at least one pharmaceutically-acceptable adjuvant, diluent or carrier.
